(19) United States
(12) Patent Application Publication
(10) Pub. No.: US 2007/0009517 A1
(43) Pub. Date: Jan. 11, 2007

(54) Method of inducing immune tolerance

(76) Inventors: Mark De Boer, Utrecht (NL); Louis Boon, Amsterdam (NL)

Correspondence Address: DARBY & DARBY P.C., P. O. BOX 5257, NEW YORK, NY, 10150-5257, US

(21) Appl. No.: 10/569,390

(22) PCT Filed: Aug. 25, 2004
(86) PCT No.: PCT/NL04/00595

§ 371 Date: Feb. 22, 2006

Related U.S. Application Data
(60) Provisional application No. 60/498,259, filed on Aug. 25, 2003.

(51) Int. Cl. A61K 39/395; A61K 38/13; A61K 31/573; A61K 31/5377; A61K 31/522; A61K 31/4745

(52) U.S. Cl. 424/144.1; 514/11; 514/233.2; 514/291; 514/171; 514/263.31

(57) ABSTRACT

Methods for inducing tolerance to a transplant in a subject are disclosed. The methods comprise administering multiple doses of a therapeutically effective amount of a CD40 antagonist alone or in combination with a CD86 antagonist, wherein the first dose of the antagonist is given before or at the time of transplantation; and administering multiple doses of a therapeutically effective amount of an immunosuppressive drug, wherein the first dose of the immunosuppressive drug is given at least several days after transplantation.

### BACKGROUND OF THE INVENTION

The first step leading to the initiation of an immune response is the recognition of antigen fragments presented in association with major histocompatibility complex (MHC) molecules. Recognition of antigens can occur directly when the antigens are associated with the MHC on the surface of foreign cells or tissues, or indirectly when the antigens are processed and then associated with the MHC on the surface of professional antigen presenting cells (APC). Resting T lymphocytes that recognize such antigen-MHC complexes become activated via association of these complexes with the T cell receptor (Jenkins et al., J. Exp. Med. 165, 302-319, 1987; Mueller et al., J. Immunol. 144, 3701-3709, 1990). If T cells are only stimulated through the T cell receptor, without receiving an additional costimulatory signal, they become nonresponsive, anergic, or die, resulting in downmodulation of the immune response, and tolerance to the antigen. (Van Gool et al., Eur. J. Immunol. 29(8):2367-75, 1999; Koenen et al., Blood 95(10):3153-61, 2000). However, if the T cells receive a second signal, termed costimulation, T cells are induced to proliferate and become functional (Lenschow et al., Annu. Rev. Immunol. 14:233, 1996).

Activated T cells express high levels of CD154 (CD40L). The cell surface expression of CD154 is tightly regulated and its biological activity is mediated by binding of the extracellular region of CD154 with CD40 on APC. In normal allogeneic recognition, CD154/CD40 interaction leads to upregulation of the B7 molecules, CD80 and CD86, Class I and Class II MHC, as well as various cytokines (Caux et al., J. Exp. Med. 180:1263, 1994) resulting in additional T cell activation, B cell proliferation and induction of antibody secretion. Therefore, the CD154/CD40 interaction can be considered as a major costimulatory signal for the activation of immune responses.

Members of the B7 family of proteins, B7-1 (CD80) and B7-2 (CD86), expressed on APCs are also critical costimulatory molecules (Freeman et al., J. Exp. Med. 174:625, 1991; Freeman et al., J. Immunol. 143:2714, 1989; Azuma et al., Nature 366:76, 1993; Freeman et al. Science 262:909, 1993). CD86 appears to play a predominant role during primary immune responses, while CD80, which is upregulated later in the course of an immune response, may be important in prolonging primary T cell responses or costimulating secondary T cell responses (Bluestone, Immunity 2:555, 1995). Moreover, the receptor to which a B7 molecule binds, such as CD28 or an inhibitory receptor such as CTLA-4, dictates whether the resulting signal to the immune cell is costimulation or inhibition. Both CD80 and CD86 exhibit binding affinity for both the costimulatory receptor CD28 and the inhibitory receptor CTLA4 (CD152). CD28 is constitutively expressed on the majority of T cells, and binding of CD86 and/or CD80 to this receptor induces the expression of anti-apoptotic proteins, stimulates growth factor and cytokine production and promotes T cell proliferation and differentiation. In contrast, CD152 is only expressed following T cell activation (Brunet, J. F., et al., 1987 Nature 328, 267-270), and the interaction of CD86 and CD80 with CD152 appears to be critical for the down-regulation of T cell responses (Waterhouse et al., Science 270:985, 1995; Allison and Krummel, Science 270:932, 1995). Further, the different expression patterns of the two receptors through the course of T cell activation is thought important for appropriate regulation of the T cell response, since the B7 molecules have a higher affinity for CD152 than for CD28 (Linsley, P. S., et al., 1991 J. Exp. Med. 174, 561-569). Thus, low CD80/CD86 expression levels results in CD152 ligation and dampening of T-cell responses, while high expression levels of CD80/CD86 results in ligation to both CD152 and CD28 resulting in T cell activation and costimulation.

Current clinical strategies for general long-term immunosuppression in disorders associated with an undesired immune response (e.g., graft rejection) are based on the long-term administration of broad acting immunosuppressive drugs, for example, signal 1 blockers such as for example cyclosporin A (CsA), FK506 (tacrolimus) and corticosteroids. However, while these immunosuppressive regimens have led to a dramatic reduction of the incidence of acute rejection episodes, they have yet to achieve a similar effect for chronic rejection or chronic/sclerosing allograft nephropathy (CAN), which is still the leading cause of graft loss during long-term follow-up. In addition, the high doses of these drugs required immediately after transplantation can be toxic to many patients leading to damage of the transplanted tissue or organ. In addition, long-term use of high doses of these drugs can also have toxic side-effects. Moreover, even in those patients that are able to tolerate these drugs, the requirement for life-long immunosuppressive drug therapy carries a significant risk of severe side effects, including tumors, serious infections, nephrotoxicity and metabolic disorders (Penn 2000; Fishman et al. 1998).

A number of recent studies have explored the effects of antibodies and fusion proteins that bind to various members of the B7 family and/or their ligand molecules on the induction of tolerance in allograft recipients. For example, it was recently demonstrated in non-human primates that a combination of anti-CD80/CD86 treatment in renal allograft recipients does not lead to the induction of tolerance. While treatment with the murine CD80/CD86 antibodies prolonged graft survival, even humanized monoclonal antibodies were not able to induce stable tolerance in all recipients (Ossevoort et al., 1999; Kirk et al. 2001; Hausen et al. 2001). Treatment with CTLA4-Ig also blocked CD80 and CD86, but was not very effective in prolonging graft survival (Kirk et al. 1997).

A number of other studies have examined the effects of antibodies to CD40 and/or CD154 on activation of the immune system. For example, the use of a humanized antagonistic anti-CD154 mAb (hu5c8) alone, in combination with CTLA4-Ig, or in combination with anti-CD80 and CD86 antibodies in rhesus kidney (Kirk et al 1997, Kirk et al. 1999), rhesus heart (Pierson et al. 1999), rhesus pancreatic islet transplantation (Kenyon et al 1999-a) or pancreatic islet transplantation in baboons (Kenyon et al 1999-b). These studies led to long survival times in most monkeys, in many cases long after cessation of treatment. Long-term kidney allograft recipients treated with hu5c8 alone lost their donor-specific MLR reactivity, but remained capable of forming donor-specific antibodies and graft infiltrating lymphocytes (Kirk et al 1999). However, trials with hu5c8 in human renal allograft recipients were aborted after reports of thromboembolic events in autoimmune studies conducted simultaneously (Knechtle et al. 2001), as activated platelets also express CD154. In addition, the hu5c8 seemed less effective in human kidney recipients than in non-human primates.

Recently, it was observed that treatment of rhesus monkey kidney allograft recipients with a combination of anti-CD80, anti-CD86 and anti-CD154 delayed the development of anti-donor antibodies, although survival times were not significantly prolonged over anti-CD154 treatment alone (Montgomery et al. 2001). Another recent study reported that blocking costimulation by anti-CD40 or anti-CD40 plus anti-CD86 prevented graft rejection in rhesus monkey allograft recipients for the duration of the treatment, but was unable to sustain graft acceptance once treatment was terminated (Haanstra et al. 2003).

Accordingly, there is a need for improved therapeutic approaches that are capable of efficiently inducing long-term immune tolerance to grafts without the need for administration of high initial doses of immunosuppressive drugs, such as signal 1 blockers, that are toxic to many patients. The successful induction of immune tolerance would further obviate the need for long-term administration immunosuppressive drugs, thereby reducing not only the costs of such treatments, but also the risk of cancer and infection in graft recipients subjected to long-term immunosuppressive therapies.

### SUMMARY OF THE INVENTION

The present invention provides improved therapies for inducing tolerance to a transplant in a subject, without the need for initial administration of toxic immuno-suppressive drugs. Immune tolerance is induced by administering a CD40 antagonist, alone or in combination with an antagonist to another costimulatory molecule (e.g., CD86), followed by administration of immunosuppressive drugs to inhibit T cell costimulation and thereby induce T cell tolerance. Using this treatment regimen, long-term tolerance beyond that previously achieved, and preferably in the absence of continued immunosuppressive drug therapy, can be achieved.

Therapeutic methods of the invention provide the significant advantages of allowing for delayed administration of immunosuppressive drugs following transplantation, and at dosages below those administered in prior immunosuppressive drug therapies. Accordingly, the invention avoids the need for administering high initial doses of broad-based immunosuppressive drugs that are currently used, and which are toxic to most patients and/or which cause secondary diseases as a result of extensive and extended immunosuppression.

Accordingly, in one embodiment, the invention provides a method for inducing tolerance to a transplant in a subject (e.g., a human) by administering a therapeutically effective amount of an antagonist to a first costimulatory molecule that is CD40, alone or in combination with an antagonist to a second costimulatory molecule, such as CD86. The initial dose of the antagonist is given before or at the time of transplantation, followed by administration of a therapeutically effective amount of an immuno-suppressive drug several days (e.g., at least about 5 days up to 8 weeks) after transplantation. Multiple doses of the antagonist and immunosuppressive drug are then continuously administered sufficient to achieve long-term tolerance without high toxicity to the subject.

The CD40 antagonist alone or in combination with the CD86 antagonist can be administered to the subject using any suitable route of administration known in the art, such as injection or i.v. infusion, for a period of time sufficient to tolerize T cells to the transplant. In particular embodiments, the antagonist is administered over a period of about 6-12 weeks, or 12 weeks up to about 6 months, after the initial dose. In yet other embodiments, the antagonist is administered to the transplant (e.g., organ or tissue) ex vivo prior to transplantation (e.g., by perfusion), followed by in vivo administration (to the recipient subject) after transplantation.

Suitable dosage regiments for the antagonist include those sufficient to maintain inhibition of CD40 and CD86-mediated costimulation within the subject until T cells are tolerized to the transplant. This can be judged, for example, by the lack of any symptoms associated with rejection. For example, suitable dosages include those that achieve initial and/or continuous serum levels of the antagonist of at least about 10-300 μg/ml, more preferably at least about 100-300 μg/ml, and more preferably at least about 100-250 μg/ml. The dosages also can be tapered during the treatment period. By tapered dosage or tapered administration is understood administration of multiple doses in decreasing amounts, i.e. wherein an individual dose is equal to or lower than a preceding dose, and at least two individual doses are lower than their preceding ones.

As with the antagonist, the immunosuppressive drug can be administered using any suitable route of administration known in the art (e.g., orally, by injection or i.v. infusion). Preferably, the first dose of the immunosuppressive drug is not administered until at least about 2, 3, 4 or 5 days, more preferably at least about 1 week, more preferably at least about 2 weeks, e.g. at least 3 weeks, 4 weeks, 6 weeks or even 8 weeks ore more after transplantation, at which point T cells have been fully or partially tolerized due to the antagonist treatment. In a particular embodiment, the initial dose of the immunosuppressive drug is not administered until completion of the administration (e.g., final dose) of the antagonist (i.e., CD40 antagonist alone or in combination with a CD86 antagonist), but during a period where serum levels of the antagonist still remain. In another embodiment, the initial dose of immunosuppressive drug is delayed until the onset of transplant rejection, for example, upon appearance of at least one symptom of rejection (e.g., in kidney transplantation, the rise of serum creatine and urea levels, as well as other rejection markers).

The immunosuppressive drug can be administered for a period of time until tolerance to the transplant is achieved in the absence of the antagonist or the immuno-suppressive drug. For example, the immunosuppressive drug can be administered over a period of about 5 days to 26 weeks (6 months), e.g. 2-12 weeks or 4-8 weeks. Alternatively, the immunosuppressive drug can be administered for longer periods of approximately 6-12 months, 12-24 months or longer. Preferably, the dosage of the immunosuppressive is tapered over the treatment period. For example, the initial dose of immunosuppressive drug can be administered for a first period (e.g. 1-4 weeks) followed by a 50% reduction in the dose for a second period of e.g. 4-8 weeks, and a further 50% reduction in the dose for a third period. The immunosuppressive drug initially can be administered at dosages routinely used in the clinic, or preferably even lower dosages that are still sufficient to maintain tolerance and prevent graft rejection, and then tapered over the course of time. For example, CsA can be administered at a dose sufficient to achieve an initial serum concentration level of about 300-500 ng/ml, followed by a serum concentration level of about 200 ng/ml, followed by a serum concentration level of about 100 ng/ml.

Suitable CD40 antagonists and CD86 antagonists that can be employed in the methods of the invention include those that interfere with the ability of these molecules to bind to their co-receptor (e.g., CD154 and CD28, respectively) and which inhibit CD40 and CD86-mediated costimulation, e.g., as measured by cytokine production and/or T cell proliferation. Exemplary antagonists include blocking antibodies and bispecific antibodies, soluble fusion polypeptides (e.g., CD86-Ig and/or CD40-Ig fusions and CD154-Ig and/or CD28-Ig fusions), peptides, peptidomimetics, nucleic acids, small molecules and the like.

In a particular embodiment, the antagonist is an antibody against CD40, CD86 and/or their respective co-receptors. Suitable antibodies can be derived from any species (e.g., human, murine, rabbit, etc.) and/or can be engineered and expressed recombinantly (e.g., chimeric, humanized and human antibodies). The antibodies can be whole antibodies or antigen-binding fragments thereof including, for example, Fab, F(ab′)₂, Fv and single chain Fv fragments. The antibodies can also include antagonistic bi-specific antibodies that bind to both CD40 and CD86, or to CD40 or CD86 and a second target molecule. In a particular embodiment, the CD40 antagonist is the chimeric anti-CD40 antibody, ch5D12, or a functionally equivalent antibody. In another particular embodiment, the CD86 antagonist is the chimeric anti-CD86 antibody, chFun-1, or a functionally equivalent antibody.

Suitable immunosuppressive drugs for use in the present invention include those known in the art that are currently used for clinical immunosuppression following transplantation. These include, for example, signal 1 blockers, steroids and other drugs. Exemplary immunosuppressive drugs include, but are not limited to, cyclosporine (CsA), tacrolimus (FK506), azathioprine, corticosteroids (e.g., prednisone), mycophenolate mofetil (MMF), rapamycin, anti-CD3 antibodies (e.g., OKT3), anti-CD25 antibodies, and rapamycin. Combinations of two or more immunosuppressive drugs also can be used. In a particular embodiment, the immunosuppressive drug is a signal-1 blocker, e.g., cyclosporine, FK506, rapamycin and MMF.

The therapeutic method of the invention can be used to induce tolerance to a wide variety of transplanted tissues and organs. Accordingly, the method can be used for broad-based treatment and/or prevention of transplant rejection. Exemplary transplants (i.e., grafts) include allografts, autografts, isografts and xenografts of organs (e.g., kidney, liver, heart and lung), tissues (e.g., bone, skeletal matrix, skin) and cells (e.g., bone marrow, stem cells).

Other features and advantages of the instant invention will be made apparent from the following detailed description and examples which should not be construed as limiting. The contents of all references, patents and published patent applications cited throughout this application are expressly incorporated herein by reference.

### BRIEF DESCRIPTION OF THE DRAWINGS

FIG. 1 is a Kaplan Meyer plot of the time to rejection (as measured by the first day serum creatinine is significantly increased) of all animals in groups 1 and 2. ▴ indicate the animals treated with the combination of ch5D12 and chFun-1 (group 2). ● indicate the animals with a low level of ch5D12 (group 1a). ▪ indicate the animals with a high level of ch5D12 (group 1b).

FIG. 2 is a Kaplan Meyer plot of the time to rejection (as measured by the first day serum creatinine is significantly increased) of all animals in groups 2 and 3. ▴ indicate the animals treated with the combination of ch5D12 and chFun-1 (group 2). □ indicate the animals treated with ATG and the combination of ch5D12 and chFun-1 (group 3).

FIG. 3 is a Kaplan Meyer plot of the time to rejection (as measured by the first day serum creatinine is significantly increased) of all animals in groups 3 and 4. ⋄ indicates untreated animals. □ indicate animals treated with ch5D12 and chFun-1 pretreated with ATG (group 3). * indicate animals treated with ch5D12 and ch-Fun-1 followed by CsA (group 4).

FIG. 4 is a bar graph depicting the incidence of rejection seen in day 21 (FIG. 4A)) and day 42 (FIG. 4B) biopsies treated with (a) ch5D12 and chFun-1; (b) high dose ch5D12; or (c) CsA for 35 days (day 35 biopsies in both panels). FIGS. 4C and 4D are bar graphs depicting the same data expressed as the mean biopsy scores for each group.

FIG. 5 is a graphic representation of CD40 expression analyzed using an anti-CD40 antibody that did not compete with 5D12 for binding to CD40 and using 5D12/FITC.

FIG. 6 is a graphic representation of CD86 expression analyzed using an anti-CD86 antibody that did not compete with 5D12 for binding to CD86 and using Fun-1/FITC.

FIG. 7 is a graphic representation showing the levels of CD8+ and CD4+ T cells following transplantation in animals (group 3) treated with anti-CD40+anti-CD86 (high dose) and ATG.

FIG. 8 is a bar graph showing latent TGF-β development after treatment with anti-CD40, anti-CD40/CD86, and anti-CD40/CD86+Cyclosporin A.

### DETAILED DESCRIPTION OF THE INVENTION

Therapeutic methods of the present invention have been shown to successfully induce immune tolerance and long-term survival in a primate model of allograft transplantation for periods not previously observed in primate transplantation models (e.g., >700 days). Moreover, long-term survival did not require the continuous administration of immunosuppressive drugs, as is used in current transplantation therapies. Accordingly, the invention provides methods for transplant therapy that provide the significant advantages of long-term transplant tolerance, without causing the toxic side effects and secondary diseases associated with current transplantation therapies.

I. Definitions

In order that the present invention may be more readily understood, certain terms are first defined below, and additional definitions are set forth throughout the Detailed Description.

As used herein, the terms “CD40” and “CD86” refer to CD40 and CD86 costimulatory molecules expressed on activated antigen presenting cells (see, for example, CD86 (B7-2) (Freeman et al. 1993 Science. 262:909 or GenBank Accession numbers P42081 or A48754); CD40 (Stamenkovic et al. EMBO 8:1403-1410, 1989 or GenBank Accession numbers CAA43045 and X60592.1), as well as fragments of CD40 and CD86 molecules, and/or functional equivalents thereof. The term “equivalent” is intended to include polypeptide sequences that have an activity of naturally occurring CD40 or CD86 molecules, e.g., the ability to bind CD40L or CD28, respectively, and modulate T cell costimulation.

As used herein, the term “CD40 antagonist” refers to agents (e.g. binding proteins, peptides and small molecules) that either inhibit functional responses mediated through CD40 signaling, or block and/or inhibit interaction of CD40 with CD40L (CD154). As used herein, the term “CD86 antagonist” refers to agents (e.g. binding proteins, peptides and small molecules) that either inhibit functional responses mediated through CD86 interaction with CD28 and/or CTLA-4 (CD152), or block and/or inhibit interaction of CD86 with CD28 and/or CTLA-4 (CD152). CD40 and CD86 antagonists also block or inhibit CD40 or CD86-mediated T cell costimulation. By blocking or inhibiting costimulatory signals, CD40 and CD86 antagonists are capable of preventing the activation of T cells and antigen presenting cells (e.g., cytokine production and T cell proliferation), thus inducing T cell anergy. A number of art recognized readouts of cell activation can be employed to measure the inhibition of T cell costimulation, such as cytokine production or T cell proliferation assays, in the presence of CD40 and/or CD86 antagonists.

As used herein, the term “immunosuppressive drug” refers to drugs (e.g., proteins, peptides, small molecules and hormones) that down-regulate an unwanted cellular and/or humoral immune response in an individual. Several immunosuppressive drugs are well known in the art and are currently used in clinical therapy including, for example, signal 1 blockers, such as cyclosporine (CsA), tacrolimus (FK506), azathioprine, corticosteroids (e.g., prednisone), mycophenolate mofetil (MMF) and rapamycin. The term “signal-1 blocker” refers to an immunosuppressive drug that interferes with T-cell receptor mediated signaling. In contrast, antagonists to CD40 and/or CD86, as well as antagonists to other costimulatory molecules, can be defined as “signal 2 blockers”. Other immunosuppressive drugs include, for example, hormones (e.g., steroids) and antibodies, such as anti-CD3 antibodies (e.g., OKT3) and anti-CD25 antibodies.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages. Immune cells involved in the immune response include lymphocytes, such as B cells and T cells (CD4⁺, CD8⁺, Th1 and Th2 cells); antigen presenting cells (e.g., professional antigen presenting cells such as B lymphocytes, monocytes, dendritic cells, Langerhans cells, and non-professional antigen presenting cells such as keratinocytes, endothelial cells, astrocytes, fibroblasts, oligodendrocytes); natural killer cells; myeloid cells, such as monocytes, macrophages, eosinophils, mast cells, basophils, and granulocytes.

As used herein, the term “anergy” or “tolerance” refers to insensitivity of T cells to T cell receptor-mediated stimulation. Such insensitivity is generally antigen-specific and persists after exposure to the tolerizing antigen has ceased. For example, anergy in T cells (as opposed to unresponsiveness) is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. 1992 Science. 257:1134).

As used herein, the term “graft” or “transplant” refers to an organ, tissue, or cell that has been transplanted from one subject to a different subject, or transplanted within the same subject (e.g., to a different area within the subject). Organs such as liver, kidney, heart or lung, or other body parts, such as bone or skeletal matrix, tissue, such as skin, intestines, endocrine glands, or progenitor stem cells of various types, are all examples of transplants. The graft or transplant can be an allograft, autograft, isograft or xenograft. The term “allograft” refers to a graft between two genetically non-identical members of a species. The term “autograft refers to a graft from one area to another on a single individual. The term “isograft” or “syngraft” refers to a graft between two genetically identical individuals. The term “xenograft” refers to a graft between members of different species.

As used herein, the term “acute rejection” refers to onset of a primary immune response to a graft, generally within days or weeks, and up to about 6 to 12 months, after transplantation. The immune response is caused by T cell recognition of the transplanted tissue associated with e.g., prominent local cytokine production, widespread pro-inflammatory activation of vascular endothelia, intense leukocyte infiltration, and development of graft-reactive, cytolytic T cells (CTL) that has traditionally been associated with the acute loss of graft function. “Hyperacute rejection” is a type of rejection that occurs very rapidly, resulting in necrosis of the transplanted tissue within minutes or a few hours of contact, and is caused by reactivity of the donor cells with pre-existing antibody.

As used herein, the terms “chronic rejection” refers to indolent, progressive immune responses that often occur one or more years after transplantation. Chronic rejection usually manifests in vascularized solid organ allografts as obliterative arteriopathy or graft vascular disease (GVD), infiltration of immunocytes, interstitial and tubularatrophy, graft arteriosclerosis, and a marked fibrosis. “Graft versus host reaction (GVH),” as used herein, refers to the pathologic consequences of a response initiated by transplanted immunocompetent T lymphocytes into an allogeneic, immunologically incompetent host. The host is unable to reject the grafted T cells and the transplanted T lymphocytes attack the tissues of the recipient due to recognition of recipient's Ags on recipient's MHC molecules (not necessarily by recipient's tissues).

As used herein, the phrase “long-term tolerance” refers tolerance (i.e., absence of rejection) of a graft or transplant in a subject for an extensive period of time, such as one or more years, preferably several years, and more preferably life. Complete tolerance occurs when tolerance is achieved and immunosuppressive treatment is no longer necessary.

II. Antagonists to CD40 and Other Costimulatory Molecules

A variety of antagonists to CD40 and other costimulatory molecules, such as CD86, are known in the art and can be employed in the therapeutic methods of the present invention.

Cell-to-cell signal exchange during antigen presentation deeply influences the profile and extent of the immune response. Together with the TCR/MHC-mediated signal, accessory signals are provided to the T cell by the antigen-presenting cell (APC), through specific receptor-ligand interactions that represent indispensable costimulation for T-cell activation and survival. The main costimulatory pathways are the B7 family members and the CD40-CD154 receptor-ligand pair. B7-1 and B7-2 costimulate T-cells by binding to CD28. Their binding is prevented by the neoexpression of CTLA-4, a CD28 homologue that can deliver a negative signal. Another CD28-like molecule, called ICOS (inducible costimulator), has been described and binds B7RP-1, a third member of the B7 family, but not B7-1 and B7-2. The CD40-CD154 interaction works as a two way costimulatory system by triggering activation signals to both T-cell and APCs. Its importance is highlighted by the discovery that mutations of the CD154 gene are responsible for a severe human immunodeficiency. Thus, disruption of the natural costimulatory interaction has can be highly effective for prevention and treatment of transplant rejection.

Accordingly, suitable antagonists for use in the invention include those that block or inhibit the interaction of CD40 with its respective co-receptors, CD40L. Suitable antagonists to other costimulatory molecules include those that antagonize the interaction (i.e., costimulation pathway) between CD86 and CD28; OX40L and OX40; LIGHT and LIGHT-L; 4-1BBL and 4-1BB (CD137); CD80 and CTLA-4 (CD152), ICOS-L and ICOS, and SLAM-L and SLAM (see e.g., Am. J. Respir. Crit. Care Med. (2000) 162(4): 164-168; J. Nephrol. (2002), 15: 7-16).

Such antagonists can be identified by a number of art recognized APC- and/or T-cell function assays, such as those described herein (e.g., T cell proliferation and/or effector function, antibody production, cytokine production, and phagocyctosis).

Agents that block CD86 and/or CD40, also can be derived using CD40 and CD86 nucleic acid or amino acid sequences. The nucleotide and amino acid sequences of these costimulatory molecules are known in the art and can be found in the literature or on a database such as GenBank. See, for example, CD86 (B7-2) (Freeman et al. 1993 Science. 262:909 or GenBank Accession numbers P42081 or A48754); CD40 (Stamenkovic et al. EMBO 8:1403-1410, 1989 or GenBank Accession numbers CAA43045 and X60592.1).

A. Antagonistic Antibodies

In a particular embodiment, the invention employs antagonistic antibodies to inhibit CD40 and/or CD86 function. As used herein, the term “antibody” includes whole antibodies or antigen-binding fragments thereof including, for example, Fab, F(ab′)₂, Fv and single chain Fv fragments. Suitable antibodies include any form of antibody, e.g., murine, human, chimeric, or humanized and any type antibody isotype, such as IgG1, IgG2, IgG3, IgG4, IgM, IgA1, IgA2, IgAsec, IgD, or IgE isotypes.

Antibodies which specifically bind CD40 or its respective co-receptor, CD40L, to prevent CD40/CD40L interaction (e.g., CD40/CD40L-mediated signaling), can be used as CD40 antagonists in the present invention. Antibodies against other costimulatory molecules as described above, such as CD86 or its respective co-receptor, CD28, also can be used in the present invention. As used herein, “specific binding” refers to antibody binding to a predetermined antigen. Typically, the antibody binds with a dissociation constant (K_{D}) of 10⁻⁷ M or less, and binds to the predetermined antigen with a K_{D} that is at least two-fold less than its K_{D} for binding to a non-specific antigen (e.g., BSA, casein) other than the predetermined antigen or a closely-related antigen.

Several CD86 antibodies are well known (see, for example, U.S. Pat. No. 5,869,050; Powers G. D., et al. (1994) Cell. Immunol. 153, 298-311; Freedman, A. S. et al. (1987) J. Immunol. 137:3260-3267; Freeman, G. J. et al. (1989) J. Immunol. 143: 2714-2722; Freeman, G. L. et al. (1991) J. Exp. Med. 174:625-631; Freeman, G. J. (1993) Science 262:909-911; WO 96/40915), and are also commercially available, e.g. from R&D Systems (Minneapolis, Minn.) and Research Diagnostics (Flanders, NJ). In a particular embodiment, the CD86 antibody used in the invention is Fun-1, or a functional equivalent thereof (Nozawa et al., J. Pathol. 169(3):309-15, 1993; Engel et al., Blood 84(5):1402-7, 1994). Several CD40 antibodies are also well known and readily available (see, for example, U.S. Pat. No. 5,677,165). In a particular embodiment, the CD40 antibody used in the invention is 5D12, or functional equivalents thereof (DeBoer et al. (1992) J. Immunol. Methods 152(1):15-23).

The heavy and light chain variable sequences for Fun-1 and 5D12 are known, as are antagonistic bispecific antibodies comprising the binding regions of both Fun-1 and 5D12 (see e.g., US 2002/0150559).

Alternatively, antagonistic CD86 and CD40 antibodies can be produced according to well known methods for antibody production. For example, antigenic peptides of CD40, CD86 or their respective ligand or receptor, which are useful for the generation of antibodies can be identified in a variety of manners well known in the art. For example, useful epitopes can be predicted by analyzing the sequence of the protein using web-based predictive algorithms (BIMAS & SYFPEITHI) to generate potential antigenic peptides from which synthetic versions can be made and tested for their capacity to generate CD40, CD86, CD40L or CD28 specific antibodies. Preferably, the antibody binds specifically or substantially specifically to the CD40 or CD86 molecule, or to their respective ligand or receptor, thereby inhibiting interaction of CD40/CD40L or CD86/CD28, respectively.

Antagonistic antibodies used in the present invention can be monoclonal or polyclonal. The terms “monoclonal antibodies” as used herein, refers to a population of antibody molecules that contain only one species of an antigen binding site capable of immunoreacting with a particular epitope of an antigen, whereas the term “polyclonal antibodies” refers to a population of antibody molecules that contain multiple species of antigen binding sites capable of interacting with a particular antigen. Techniques for generating monoclonal and polyclonal antibodies are well known in the art (See, e.g., Current Protocols in Immunology, Coligan et al., eds., John Wiley & Sons, http://www.does.org/masterli/cpi.html).

Recombinant antagonistic CD40 and CD86 antibodies, such as chimeric and humanized monoclonal antibodies, comprising both human and non-human portions can be made using standard recombinant DNA techniques, and are also within the scope of the invention. Such chimeric and humanized monoclonal antibodies can be produced by recombinant DNA techniques known in the art, for example using methods described in Robinson et al. International Patent Publication WO87/02671; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; U.S. Pat. No. 5,225,539 5,565,332, 5,871,907, or 5,733,743; Jones et al. (1986) Nature 321:552-525; Verhoeyan et al. (1988) Science 239:1534; and Beidler et al. (1988) J. Immunol. 141:4053-4060.

Recombinant chimeric antibodies can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

Fully human antibodies that bind to CD40, CD86 and/or their respective ligand or receptor can also be employed in the invention, and can produced using techniques that are known in the art. For example, transgenic mice can be made using standard methods, e.g., according to Hogan, et al., “Manipulating the Mouse Embryo: A Laboratory Manual”, Cold Spring Harbor Laboratory, which is incorporated herein by reference, or are purchased commercially. Embryonic stem cells are manipulated according to published procedures (Teratocarcinomas and embryonic stem cells: a practical approach, Robertson, E. J. ed., IRL Press, Washington, D.C., 1987; Zijlstra et al. (1989) Nature 342:435-438; and Schwartzberg et al. (1989) Science 246:799-803, each of which is incorporated herein by reference). For example, transgenic mice can be immunized using purified or recombinant CD40 or CD86 or a fusion protein comprising at least an immunogenic portion of the extracellular domain of CD40 or CD86. Antibody reactivity can be measured using standard methods. The term “recombinant human antibody,” as used herein, includes all human antibodies that are prepared, expressed, created or isolated by recombinant means. Such recombinant human antibodies have variable and constant regions derived from human germline immuno-globulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Single chain antagonistic antibodies that bind to CD40, CD86 or their respective ligand or receptor also can be identified and isolated by screening a combinatorial library of human immunoglobulin sequences displayed on M13 bacteriophage (Winter et al. 1994 Annu. Rev. Immunol. 1994 12:433; Hoogenboom et al., 1998, Immunotechnology 4:1). For example, CD40, CD86, CD40L or CD28 can be used to thereby isolate immunoglobulin library members that bind a CD40, CD86, CD40L or CD28 polypeptide. Kits for generating and screening phage display libraries are commercially available and standard methods may be employed to generate the scFv (Helfrich et al. J. Immunol Methods 2000, 237: 131-45; Cardoso et al. Scand J. Immunol 2000. 51: 337-44). Alternatively, Ribosomal display can be used to replace bacteriophage as the display platform (see, e.g., Hanes et al. Nat. Biotechnol. 18:1287, 2000; Wilson et al. Proc. Natl. Acad. Sci. USA 98:3750, 2001; OR Irving et al., J. Immunol. Methods. 248:31, 2001).

In yet another embodiment of the invention, bispecific or multispecific antibodies that bind to CD86 and CD40 or antigen-binding portions thereof. Such bispecific antibodies are described, for example, in US 2002/0150559, and can be generated, e.g., by linking one antibody or antigen-binding portion (e.g., by chemical coupling, genetic fusion, noncovalent association or otherwise) to a second antibody or antigen-binding portion. Bispecific and multispecific molecules of the present invention can be made using chemical techniques, “polydoma” techniques or recombinant DNA techniques. Bispecific and multispecific molecules can also be single chain molecules or may comprise at least two single chain molecules. Methods for preparing bi- and multispecific molecules are described for example in D. M. Kranz et al. (1981) Proc. Natl. Acad. Sci. USA 78:5807 and U.S. Pat. Nos. 4,474,893; 5,260,203; 5,534,254. 5,455,030; 4,881,175; 5,132,405; 5,091,513; 5,476,786; 5,013,653; 5,258,498; and 5,482,858.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or substituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the bispecific and multispecific molecule has at least one antigen binding region specific for an FcγR and triggers at least one effector function.

B. Fusion Protein Antagonists

Another form of CD40 and/or CD86 antagonist that can be employed in the methods of the present invention is a soluble form of (e.g., a fusion protein or chimeric protein) CD40, CD86, their respective co-receptors (i.e., CD40L and CD28), or fragments and variants thereof. As used herein, a CD40 or CD86 “chimeric protein” or “fusion protein” comprises a CD40 or CD86 polypeptide, fragment, or functional variant thereof, operatively linked to a non-CD40 or CD86 polypeptide. Within a CD40 or CD86 fusion protein the CD40 or CD86 polypeptide can correspond to all or a portion of a CD40 or CD86 protein. In a particular embodiment, a CD40 or CD86 fusion protein comprises at least one biologically active portion of a CD40 or CD86 protein, e.g., the extracellular domain of a CD40 or CD86 protein which binds to co-receptor. Within the fusion protein, the term “operatively linked” is intended to indicate that the CD40 or CD86 polypeptide and the non-CD40 or CD86 polypeptide are fused in-frame to each other. The non-CD40 or CD86 polypeptide can be fused to the N-terminus or C-terminus of the CD40 or CD86 polypeptide.

A CD40 or CD86 fusion protein can be produced by recombinant expression of a nucleotide sequence encoding a first peptide having CD40 or CD86 activity and a nucleotide sequence encoding second peptide according to standard techniques (e.g., see Current Protocols in Molecular Biology, eds. Ausubel et al. John Wiley & Sons: 1992). Preferably, the first peptide consists of a portion of the CD40 or CD86 polypeptide (e.g., a portion after cleavage of the signal sequence) that is sufficient to modulate an immune response. The second peptide can include an immunoglobulin constant region, for example, a human Cγ1 domain or Cγ4 domain (e.g., the hinge, CH2 and CH3 regions of human IgCγ1, or human IgCγ4 (see e.g., Capon et al. U.S. Pat. Nos. 5,116,964; 5,580,756; 5,844,095); a GST peptide, or an influenza hemagglutinin epitope tag (HA) (e.g. Herrsher et al., Genes Dev. 9:3067-3082, 1995).

The resulting fusion protein may have altered CD40 or CD86 solubility, binding affinity, stability and/or valency (i.e., the number of binding sites available per molecule) and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying fusion proteins and peptides are known in the art.

Particularly preferred CD40 or CD86 fusion proteins include the extracellular domain portion or variable region-like domain of a human CD40 or CD86 coupled to an immunoglobulin constant region (e.g., the Fc region). Such fusion proteins can be monovalent or bivalent as is recognized in the art. The immunoglobulin constant region may contain genetic modifications which reduce or eliminate effector activity inherent in the immunoglobulin structure. For example, DNA encoding the extracellular portion of a CD40 or CD86 polypeptide can be joined to DNA encoding the hinge, CH2 and CH3 regions of human IgGγ1 and/or IgGγ4 modified by site directed mutagenesis, e.g., as taught in WO 97/28267.

C. Peptide Antagonists

A number of useful antagonists can also be derived from CD40 or CD86 polypeptide sequences and their co-receptors. An antagonist may, for instance, be a functional variant of the naturally occurring protein (e.g., a soluble form of CD40, CD86 or their respective co-receptors), a mimic or peptidomimetic that inhibits the activity of CD40 or CD86 required for the immunosuppressive effect. Variants of the CD40 or CD86 proteins which serve as antagonists can be generated by mutagenesis (e.g., amino acid substitution, amino acid insertion, or truncation of the CD40 or CD86 protein), and identified by screening combinatorial libraries of mutants, such as truncation mutants, of a CD40 or CD86 protein for the desired activity, (e.g. CD40 or CD86 protein antagonist).

For example, a variegated library of CD40 or CD86 variants can be generated by combinatorial mutagenesis at the nucleic acid level, for example, by enzymatically ligating a mixture of synthetic oligonucleotides into gene sequences such that a degenerate set of potential CD40 or CD86 sequences is expressible as individual polypeptides, or alternatively, as a set of larger fusion proteins (e.g., for phage display) containing the set of CD40 or CD86 sequences therein. Chemical synthesis of a degenerate gene sequence can also be performed in an automatic DNA synthesizer, and the synthetic gene then ligated into an appropriate expression vector. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

Soluble forms of CD40, CD86 or their co-receptors can serve as antagonists in the methods of the invention. Such forms can be engineered using art recognized methods, and can comprise or consist of, e.g., an extracellular domain of a CD40 or CD86 protein. In one embodiment, the extracellular domain of the CD40 or CD86 polypeptide comprises the mature form of a CD40 or CD86 polypeptide, but not the transmembrane and cytoplasmic domains. A soluble form of CD40 or CD86 polypeptide, or a receptor binding portion thereof, which is multivalent to the extent that it is sufficient to crosslink the receptor is also considered an antagonist.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CD40 or CD86 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify CD40 or CD86 variants (Arkin and Youvan (1992) Proc. Natl. Acad. Sci. USA 89:7811-7815; Delagrave et al. (1993) Protein Eng. 6(3):327-331).

Once suitable peptide antagonists are identified, systematic substitution of one or more amino acids of either CD40 or CD86 amino acid sequence, or a functional variant thereof, with a D-amino acid of the same type (e.g., D-lysine in place of L-lysine) can also be used to generate a peptide agonist which has increased stability. In addition, constrained peptides comprising a CD40 or CD86 amino acid sequence, a functional variant thereof, or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

Peptides that act as antagonists CD40/CD40L or CD86/CD28 interactions can be produced recombinantly or direct chemical synthesis. Further, peptides may be produced as modified peptides, with non-peptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, and desirable pharmacokinetic properties.

Another form of antagonist is a peptide analog or peptide mimetic of the CD40 or CD86 protein. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference) and are usually developed with the aid of computerized molecular modeling. Peptide mimetics that are structurally similar to CD40 or CD86 or functional variants thereof, can be used to produce an antagonistic effect. Generally, peptidomimetics are structurally similar to the paradigm polypeptide (CD40 or CD86) but have one or more peptide linkages (—CO—NH—) optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH₂NH—, —CH₂CH₂—); Spatola, A. F. et al. (1986) Life Sci. 38:1243-1249 (—CH₂—S); Hann, M. M. (1982) J. Chem. Soc. Perkin Trans. I. 307-314 (—CH═CH—, cis and trans); Almquist, R. G. et al. (190) J. Med. Chem. 23:1392-1398 (—COCH₂—); Jennings-White, C. et al. (1982) Tetrahedron Lett. 23:2533 (—COCH₂—); Szelke, M. et al., EP 45665 (1982) CA: 97:39405 (1982) (—CH(OH)CH₂—); Holladay, M. W. et al. (1983) Tetrahedron Lett. (1983) 24:4401-4404 (—C(OH)CH₂—); and Hruby, V. J. (1982) Life Sci. (1982) 31:189-199 (—CH₂—S—); each of which is incorporated herein by reference.

D. Nucleic Acid Antagonists

Nucleic acid molecules can also be used as antagonists of CD40 or CD86 activity. For example, isolated nucleic acid molecules that are antisense molecules can be used as modulating agents to inhibit CD40 and/or CD86 expression. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire CD40 or CD86 coding strand, or only to a portion thereof. In one embodiment, an antisense nucleic acid molecule is antisense to a “coding region” of the coding strand of a nucleotide sequence encoding CD86 or CD40. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding CD40 or CD86. The term “noncoding region” refers to 5′ and 3′ sequences which flank the coding region that are not translated into amino acids (i.e., also referred to as 5′ and 3′ untranslated regions).

Given the coding strand sequences encoding CD40 and CD86 disclosed in the art, antisense nucleic acids can be designed according to the rules of Watson and Crick base pairing. The antisense nucleic acid molecule can be complementary to the entire coding region of CD40 or CD86 mRNA, but preferably is an oligonucleotide which is antisense to only a portion of the coding or noncoding region of CD40 or CD86 mRNA. For example, the antisense oligonucleotide can be complementary to the region surrounding the translation start site of CD40 or CD86 mRNA. An antisense oligo-nucleotide can be, for example, about 5, 10, 15, 20, 25, 30, 35, 40, 45 or 50 nucleotides in length. An antisense nucleic acid for use in the methods of the invention can be constructed using chemical synthesis and enzymatic ligation reactions using procedures known in the art. For example, an antisense nucleic acid molecule (e.g., an antisense oligonucleotide) can be chemically or recombinantly synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Alternatively, an antisense nucleic acid molecule can be an a-anomeric nucleic acid molecule, or a ribozyme. An a-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). Ribozymes are catalytic RNA molecules with ribonuclease activity which are capable of cleaving a single-stranded nucleic acid molecule, such as an mRNA, to which they have a complementary region and can be used to catalytically cleave CD40 or CD86 mRNA. Such molecules can be constructed by methods known in the art. (see, e.g., Cech et al. U.S. Pat. No. 4,987,071; and Cech et al. U.S. Pat. No. 5,116,742).

In another embodiment, CD40 or CD86 gene expression can be inhibited by targeting nucleotide sequences complementary to the regulatory region of the CD40 or CD86 (e.g., the CD40 or CD86 promoter and/or enhancers) to form triple helical structures that prevent transcription of the CD40 or CD86 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In another embodiment, a compound that promotes RNAi can be used to inhibit CD40 or CD86 expression. RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In yet another embodiment, the CD40 or CD86 nucleic acid molecules of the present invention can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; WO88/09810) or the blood-brain barrier (see, e.g. WO89/10134). In addition, oligo-nucleotides can be modified with hybridization-triggered cleavage agents (see, e.g., Krol et al. (1988) Biotechniques 6:958-976) or intercalating agents (see, e.g., Zon (1988) Pharm. Res. 5:539-549). To this end, the oligonucleotide can be conjugated to another molecule, (e.g., a peptide, hybridization triggered cross-linking agent, transport agent, or hybridization-triggered cleavage agent).

III. Immunosuppressants

Immunosuppressive drugs suitable for use in the present invention include agents that down-regulate and/or suppress an immune response in a subject, for example, by blocking or inhibiting the activation or proliferation of T cells. Such drugs are well known in the art and are readily available through commercial sources (see e.g., Immunobiology, Vol. 5 (chapter 14), ©2001 Garland Publishing, New York, N.Y., the contents of which are incorporated by reference herein). In addition, such drugs are routinely used in current clinical therapies and, as such, are easily adaptable to the methods of the present invention.

Transplant rejection is caused by detrimental immune responses against tissue antigens. Thus, the goal of immunosuppressive drug therapy is to down-regulate such immune responses to avoid damage to the tissues or disruption of their function. In the methods of the present invention, this is used in combination with therapies that induce T cell anergy or tolerance (e.g., anti-CD40 therapy alone or in combination with anti-CD86 therapy) to the tissues. This achieves effective, long-term prevention of transplant rejection.

Accordingly, a wide variety of known immunosuppressive drugs can be used in the methods of the invention. Drugs currently used in the clinic to suppress the immune system can be divided into three categories. First, anti-inflammatory drugs of the corticosteroid family, such as prednisone, are used. Second, cytotoxic drugs, such as azathioprine and cyclophosphamide, are used. Third, fungal and bacterial derivatives, such as cyclosporin A (CsA), FK506 (tacrolimus), and rapamycin (sirolimus), which inhibit signaling events within T lymphocytes, are used. Specifically, these fungal and bacterial derivatives exert their biological effects by binding to intracellular immunophilins, forming complexes that interfere with signaling pathways important for the clonal expansion of T lymphocytes.

The foregoing immunosuppressive drugs are all very broad in their actions and inhibit protective functions of the immune system as well as harmful ones. Thus, it is well known that opportunistic infection is a common complication of immuno-suppressive drug therapy.

In a particular embodiment, the immunosuppressive drug used in the methods of the present invention is a signal 1 blocker, such as cyclosporine (CsA), FK506, azathioprine, a corticosteroid, mycophenolate mofetil (MMF) and/or rapamycin. In other embodiments, the immunosuppressive drug is a hormone (e.g., a steroid) or an antibody, such as anti-CD3 antibodies (e.g., OKT3) and anti-CD25 antibodies.

IV. Therapeutic Compositions

CD40 and/or CD86 antagonists can be formulated, separately or together, with a variety of pharmaceutically acceptable carriers prior to administration. Similarly, immunosuppressive drugs can be formulated with a variety of pharmaceutically acceptable carriers prior to administration. As used herein, “pharmaceutically acceptable carriers” include any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

Suitable pharmaceutically acceptable carriers include sterile aqueous solutions or dispersions and sterile powders for the extemporaneous preparation of sterile injectable solutions or dispersion, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, mono-stearate salts and gelatin. Supplementary active compounds can also be incorporated into the compositions. Carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxyalkanoic acids, aromatic acids, aliphatic and aromatic sulfonic acids and the like. Base addition salts include those derived from alkali and alkaline earth metals, such as sodium, potassium, magnesium, calcium and the like, as well as from nontoxic organic amines, such as N,N′-dibenzylethylenediamine, N-methyl-glucamine, chloroprocaine, choline, diethanolamine, ethylenediamine, procaine and the like.

The pharmaceutical compositions used in the methods of the present invention can be administered by a variety of methods known in the art. As will be appreciated by the skilled artisan, the route and/or mode of administration will vary depending upon the desired results. For example, pharmaceutical formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent.

Formulations of the present invention that are suitable for injection must be sterile and fluid to the extent that the composition is deliverable by syringe. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable composition can be brought about by including an agent which delays absorption, for example, aluminum monostearate or gelatin. Sterile injectable solutions can be prepared by incorporating the active compound in the required amount in an appropriate solvent with one or a combination of ingredients enumerated above, as required, followed by sterilization microfiltration. In the case of sterile powders for the preparation of injectable solutions, the preferred methods of preparation are vacuum drying and freeze-drying (lyophilization) that yield a powder of the active ingredient.

Formulations of the present invention which are suitable for the topical or transdermal administration of compositions of this invention include powders, sprays, ointments, pastes, creams, lotions, gels, solutions, patches and inhalants. Dosage forms for vaginal administration also include pessaries, tampons, creams, gels, pastes, foams or spray formulations containing such carriers as are known in the art to be appropriate. The active compound may be mixed under sterile conditions with a pharmaceutically acceptable carrier, and with any preservatives, buffers, or propellants which may be required. Alternatively, when the active compound is suitably protected, as described above, the compound may be orally administered, for example, with an inert diluent or an assimilable edible carrier.

The pharmaceutical compositions of the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

V. Therapeutic Dosage Regimens

A variety of therapeutic dosage regimens can be employed in the methods of the present invention according to the guidelines described below. In all cases, the regimen involves administering to a subject, prior to or at the time of transplantation, a therapeutically effective amount of an antagonist of CD40 alone or in combination with and an antagonist of CD86, followed (for example, at least several days later) by administration of a therapeutically effective amount of an immunosuppressive agent(s).

The term “therapeutically effective” amount, as used herein, refers to a dosage of antagonist or immunosuppressive drug that induces complete or substantial tolerance to a transplant in a subject (e.g., 80% tolerance relative to untreated subjects). Lack of tolerance, i.e., rejection, can be measured by the development of one or more symptoms associated with graft rejection including, but not limited to, a substantial rise in serum creatine levels, reduced organ function, pain or swelling in the location of the organ or tissue, fever, and/or general discomfort. Conditions associated with graft rejection include, without limitation, acute graft rejection, graft-versus-host disease (GVHD), chronic graft rejection (e.g., chronic/sclerosing nephropathy).

Accordingly, tolerance (e.g., the prevention and/or reduction in signs and/or symptoms of acute and/or chronic transplant rejection), can be evaluated using art-recognized assays and methods known to measure the aforementioned symptoms of transplant rejection. These include, e.g., the assays and parameters described in the Examples provided below, such as those that measure serum creatine levels and donor-specific antibody levels, as well as needle biopsies etc.). Alternatively, tolerance can be evaluated by examining the reduction of T cell activation and proliferation using standard assays. One of ordinary skill in the art also can determine such therapeutically effective amounts based on factors such as the subject's size, the severity of the signs and/or subject's symptoms, and the particular composition or route of administration selected.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Accordingly, dosage regimens are adjusted to provide the optimum desired response in a subject that is sufficient to maintain the blockage or inhibition of CD40/CD40L interaction, or CD40/CD40L interaction and interaction of other costimulatory molecules, such as CD86/CD28, until graft tolerance is induced. For example, the dosage regimen can be adjusted to achieve sufficient serum levels of antagonist to achieve full coating of substantially all CD40 and/or CD86 molecules expressed and/or to inhibit or block the functional activity of substantially all CD40 and/or CD86 molecules expressed within the transplant subject. When administering both CD40 and CD86 antagonists, the antagonists can be co-administered simultaneously in the same pharmaceutically acceptable excipient, or co-administered one after the other in separate pharmaceutically acceptable excipients.

The initial administration of the CD40 and CD86 antagonists can be before transplantation (e.g., about 1 week, or 5, 4, 3, 2 or 1 day(s) prior to transplantation), at the time of transplantation (e.g., on day 0), or shortly following transplantation (e.g., 1 or 2 days after transplantation), with repeated dosages thereafter for a sufficient period to substantially tolerize T cells to the transplant. The antagonis(s) can also be administered in multiple doses (e.g., daily, every 2 days, every 3 days, once weekly or once bi-weekly, or combinations thereof, over a period of time lasting, for example, approximately 2-26 weeks, 4-16 weeks, 6-12 weeks or 8-10 weeks after the initial dose.

In particular embodiments, the initial dosage of the antagonist(s) is approximately 1 to 20 mg/kg, 1 to 10 mg/kg, or 1 to 5 mg/kg, with subsequent doses being reduced to approximately 0.1 to 10 mg/kg or 1 to 5 mg/kg. In other particular embodiments, the dosages used are sufficient to maintain an initial or continuous serum level of the antagonist(s) of at least about 10-300 μg/ml, 75-250 μg/ml, 100-250 μg/ml, 150-250 μg/ml or 100-200 μg/ml during the treatment period.

Initial dosages of the antagonist can also be administered ex vivo to the transplant prior to transplantation into the subject, followed by in vivo administration thereafter. For example, the transplant can be washed and/or perfused using well-known methods in media containing the antagonist in an amount sufficient to sufficient to saturate substantially all CD40 and/or CD86 molecules, and/or their respective ligand molecules, in the donor tissue. The transplant can then be surgically grafted into the recipient subject. The term “saturate” refers to binding to CD40 and/or CD86 molecules, and/or their respective ligand molecules, resulting in a functional antagonistic effect on the molecule. This includes, but is not limited to, inhibiting or blocking the interaction of the molecules with their respective ligands.

As with the CD40 and CD86 antagonists, the dosage regimens of the immunosuppressive drug(s) can also be adjusted to provide the optimum therapeutic benefit (e.g., long-term survival and tolerance). In all cases, the optimum dosage regimen includes the lowest amount of drug necessary to maintain anergy to the transplant following or during treatment with antagonist. This preserves the health of the transplant from toxicity and the overall strength of the recipient's immune system. In addition, the toxic effects associated with early immunosuppressive drug treatment (e.g., organ toxicity and/or systemic toxicity) are avoided by delaying administration of the immunosuppressive drug until the antagonist treatment regimen is completed or nearly completed. For example, the immunosuppressive drug(s) can be administered at least several days after transplantation, e.g., at least about 2, 3, 4 or 5 days, preferably at least about 1 week, more preferably at least about 2-8 weeks, and more preferably at least about 6-8 weeks after transplantation.

In a particular embodiment, to maximize the time before administering the immunosuppressive drug, the initial dose of the immunosuppressive drug is delayed until the final dose of the antagonist(s) (i.e., CD40 antagonist alone or in combination with a CD86 antagonist) has been administered. Alternatively, the immunosuppressive drug can be delayed until the first symptoms of acute or chronic rejection are observed, which can be prior to the final administration of the antagonists, or can be after the final administration of the antagonists, depending on the subject being treated. Accordingly, in cases where the subject does not demonstrate early signs of graft rejection (e.g., due to tolerance induced by the initial CD40/CD86 antagonist therapy), treatment with the immunosuppressive drug can be delayed as long as possible after transplantation, e.g., at least 6 to 8 weeks, in order to minimize the doses required to maintain or establish tolerance and to reduce toxicity.

The immunosuppressive drug can be administered in multiple doses (e.g., daily, every 2 days, every 3 days, once weekly, once bi-weekly or monthly) over a period of time lasting until the patient is tolerized. In certain subjects, this can be achieved in as little as 2-12 weeks, although longer periods (e.g., up to six months, one year, two years or longer after the initial dose of the drug) are required to maintain tolerance in other patients. Notwithstanding, due to the initial tolerance achieved by the CD40 and/or CD86 antagonist, the dosage levels of immunosuppressive drug required to maintain tolerance are generally expected to be significantly lower than those used in current clinical transplantation therapy.

For example, when using CsA, the immunosuppressive drug can be administered at a dosage of up to about 10 mg/kg or 1 to 5 mg/kg for a period of time of about 1 week, 2 weeks, 3 weeks or up to about 1 month, with subsequent doses being reduced to approximately 0.1 to 10 mg/kg or 1 to 5 mg/kg for the remaining treatment period. In other embodiments, CsA can be administered at a dose of about 5 to 10 mg/kg for a time period of 1 to 4 weeks, followed by a 50% reduction in the dose for a second time period of 1 to 4 weeks, and a further 50% reduction in the dose for a third period of time of 1 to 4 weeks, or up to 6 months. In other particular embodiments, CsA can be administered at dosages sufficient to achieve an initial serum concentration level of about 300-500 ng/ml for 4 about weeks, about 200 ng/ml for the following 4 weeks, and about 100 ng/ml for an additional 4 weeks.

The present invention is further illustrated by the following examples which should not be construed as further limiting.

### EXAMPLES

The following studies were performed to study the efficacy of CD40 and CD86 antagonist therapy to induce long-term immune tolerance in a rhesus monkey kidney allograft model, without the need for initial or high doses of immunosuppressive agents currently used. Primates were chosen for this study because the results obtained using this animal model are known to correlate with clinical transplantation more closely than those observed in inbred murine disease models (since non-human primates, like humans, are outbred species). In addition, monoclonal antibodies (Mabs) raised against human costimulatory molecules are cross-reactive in most non-human primate species, providing a more direct analysis of clinically relevant agents.

Specifically, treatment regimens were designed to inhibit the onset of T cell co-stimulation by blocking CD40 signaling or CD40 and CD86 signaling, while still permitting CD80 to interact with CTLA4 (CD152) thus maintaining the signals required for T-cell down-regulation. This treatment, which allows for the induction of immune tolerance to the graft, is then followed by treatment with immunosuppressive drugs to maintain inhibition of T-cell responses until tolerance is established.

The results obtained from these experiments demonstrate that delaying treatment with immunosuppressive drugs not only results in reduced toxicity, but unexpectedly, promotes long-term survival and graft tolerance, even after immunosuppressive therapy is terminated. Thus, the therapeutic approaches described herein provide the substantial advantage of avoiding the risk of serious infections and cancer associated in current daily clinical practice using life-long immunosuppressive therapies.

Materials and Methods

Animals

Naive, captive bred 4-6 kg rhesus monkeys (Macaca mulatta) were either born and raised at the Biomedical Primate Research Center (The Netherlands) (BPRC) or were purchased from a commercial breeding station. The animals were fed monkey chow supplemented by fresh fruit and vegetables, and tap water was provided ad libitum. All procedures were performed in accordance with guidelines of the Animal Care and Use Committee installed by Dutch law. All animals used in the study were in overall good health; had normal hematology and clinical chemistry values; had no history of allo-immunization; had, no history of immunization with human or murine serum component; and were MHC and ABO typed.

All animals were typed for Mamu-A, B and DR antigens by serology (Bontrop et al. 1995). Disparity for DR locus antigens was confirmed by DRB typing (Doxiadis et al. 2000). Recipients were mismatched for one or two Mamu-DR antigens, and had at least one Mamu-A and -B mismatched antigen with the donor. Total Mamu-DR mismatches were distributed equally in both groups. The recipient-donor pairs were compatible for ABO-antigens (Doxiadis et al. 1998). In addition, the stimulation index of the one-way mixed lymphocyte reaction of the recipient cells directed against the donor antigens was positive (SI>3). All animals were screened for pre-existing antibodies to ch5D12 and chFun-1 by ELISA (see Anti-Chimeric-antibody responses (RACA)).

Production and Purification of Chimeric Anti-Human Anti-CD40 and Anti-CD86

NS0 cells were transfected simultaneously by electroporation with the two expression plasmids encoding for the variable light and variable heavy chain of the anti-CD40 Mab (ch5D12) and anti-CD86 (chFun-1) Mab, respectively. For both Mabs, stable cell-lines were selected using G418 and mycophenolic acid as selection markers. Cell-lines were then screened for high production levels. A high producing cell-line for each Mab was then expanded by growth in a shaker flask and adapted to serum-free production medium. After a last quality check for each Mab, large amounts of material were obtained by growth in a bioreactor, and the Mabs were purified using protein A followed by gel-filtration.

Purified protein concentrations of both Mabs the protein was determined according to standard methods. Binding to CD86 or CD40 was then tested by ELISA, and by FACS on B cells. These analyses demonstrated equivalent binding of the chimeric Mabs compared to their respective mouse counterparts. Each of the chimeric Mab preparations was then further tested for purity and endotoxin levels and was found to meet the standards for in vivo use in non-human primates.

Kidney Transplantation

Heterotopic kidney allo-transplantation with bilateral nephrectomy was performed as described previously (Neuhaus et al. 1982, Ossevoort et al. 1999). The clinical condition of the animals was monitored by daily visual inspection and by frequent haematological and clinical chemistry blood values determined in a clinical laboratory (SSDZ, Delft) or at the BPRC. Needle biopsies (18G, BARD, The Netherlands) were taken from the kidney at 2, 6, 10, 16 and 26 weeks after transplantation. The biopsies were stored in formaline and/or cryopreserved for later analysis.

Transplant rejection was monitored by increases in serum creatinine and urea levels (Haanstra et al. 2003). A rejection episode was not treated. When serum creatinine showed a significant rise or when the clinical condition began to deteriorate, the animals were euthanized and a complete necropsy was performed in which the abdominal and thoracic cavities were opened and internal organs examined in situ and preserved in a neutral aqueous phosphate-buffered 4% solution of formaldehyde. For histological examination, biopsy material and tissues from the necropsy were formalin-fixed and paraplast-embedded, and samples of the spleen and graft were also cryopreserved.

Biopsies were analyzed by four-micron-thick sections were stained with hematoxylin and eosin (H&E), periodic acid Schiff, and a silver impregnation stain (Jones) (Haanstra et al. 2003). Histomorphological evaluation of allograft rejection was performed according to the Banff classification (Racusen et al. 1999).

Mab and Drug Treatment.

Group 1 animals (n=7) were treated with anti-CD40 alone. Two animals (Group 1a) received two initial doses of 10 mg/kg i.v. of the Mab on day −1 and day 0, followed by 5 mg/kg on days 4, 7, 11, and 14 and 5 mg/kg i.v. weekly thereafter until day 56. Circulating Mab levels in these two animals were found to be lower than 100 μg/ml serum after day 14. Therefore, the remaining animals in the first group (Group 1b) were treated with a doubling of the dosing schedule, 20 mg/kg on days −1 and 0, on days 4, 7, 11, and 14 with 10 mg/kg and with 5 mg/kg twice weekly thereafter until day 56. No additional immunosuppression or rescue medication was provided to these animals.

Group 2 animals (n=6) were treated with a combination of anti-CD40 and anti-CD86. Two animals (Group 2a) received two initial doses of 10 mg/kg i.v. for each Mab on day −1 and day 0, followed by 5 mg/kg i.v. on days 4, 7, 11, and 14 and 5 mg/kg bi-weekly thereafter until day 56. Again, circulating Mab levels were found to be lower than 100 μg/ml serum after day 14, and therefore subsequent animals (Group 2b) were treated with a doubling of the dosing schedule, 20 mg/kg on days −1 and 0, on days 4, 7, 11, and 14 with 10 mg/kg and with 5 mg/kg twice weekly thereafter until day 56. No additional immunosuppression or rescue medication was provided to these animals.

Group 3 animals were pretreated with 20 mg/kg Thymoglobuline (ATG) (Imtix-Sangstat) on day −1 (i.v.) and 10 mg/kg on day 0, followed by 10 mg/kg anti-CD40+anti-CD86 on days 4, 7, 11 and 14, and 5 mg/kg bi-weekly thereafter until day 56 to a serum level of at least 250 μg/ml. The animals were further treated with CsA from day 42-100 onward with 5-10 mg/kg i.m. In addition, the animals were treated on day −1 with 10 mg/kg Solumedrol (methylprednisolon, Pharmacia & Upjohn). After day 42 the first rejection episode in each animal was treated with 3 days of 10 mg/kg Solumedrol, and animals continued on CsA (Novartis) and 1 mg/kg prednisolon, tapering after day 90 (Nourypharma) thereafter.

Group 4 animals were treated with a combination of 20 mg/kg of anti-CD40+ anti-CD86 on days −1 and 0, 10/mg/kg of anti-CD40+anti-CD86 on days 4, 7, 11 and 14, followed by 5 mg/kg bi-weekly for 8 weeks i.v. to a serum level of at least 250 μg/ml. CsA was administered from days 42-124 orally twice weekly and i.m. five times weekly to obtain blood concentration levels of 300 ng/ml for 4 weeks, 200 ng/ml for the following 4 weeks, and 100 ng/ml for the final four weeks.

Historical controls were treated with CsA 10 mg/kg i.m. daily for thirty-five days, with detectable serum levels until day 70. A summary of the dosing schedules used is depicted in Table 1.

TABLE 1
Dosing Schedule

| | EXPERIMENTAL | |
|---|---|---|
| GROUP | TREATMENT | Dosing |
| 1a | | |
| | Anti CD40 alone | day −1, 0: 10 mg/kg; |
| | low dose | day 4, 7, 11, 14, 21, 28, 35, 42, 49, 56: 5 mg/kg |
| 1b | | day −1, 0: 20 mg/kg; |
| | Anti CD40 alone | day 4, 7, 11, 14: 10 mg/kg; |
| | high dose | day 18, 21, 25, 28, 32, 35, 39, 42, 46, 49, 52, 56: 5 mg/kg |
| 2a | Anti CD40 + | day −1, 0: 10 mg/kg; |
| | anti CD86 | day 4, 7, 11, 14, 21, 28, 35, 42, 49, 56: 5 mg/kg |
| | low dose | |
| 2b | Anti CD40 + | day −1, 0: 20 mg/kg; |
| | anti CD86 | day 4, 7, 11, 14: 10 mg/kg; |
| | high dose | day 18, 21, 25, 28, 32, 35, 39, 42, 46, 49, 52, 56: 5 mg/kg |
| 3 | Anti CD40 + | day −1, 0: 20 mg/kg; |
| | anti CD86 | day 4, 7, 11, 14: 10 mg/kg; |
| | high dose | day 18, 21, 25, 28, 32, 35, 39, 42, 46, 49, 52, 56: 5 mg/kg |
| | thymoglobuline (ATG) | day −1: 20 mg/kg i.v.; day 0: 20 mg/kg s.c. |
| | solumedrol | day −1: 10 mg/kg and after day 42 in case of rejection 3 × 10 mg/kg |
| | cyclosporine | day 42-100: 5-10 mg/kg i.m. |
| | di-adreson-F | after day 42, subsequent to solumedrol treatment: 1 mg/kg, |
| | | taper after day 90. |
| 4 | Anti CD40 + | day −1, 0: 20 mg/kg; |
| | anti CD86 | day 4, 7, 11, 14: 10 mg/kg; |
| | high dose | day 18, 21, 25, 28, 32, 35, 39, 42, 46, 49, 52, 56: 5 mg/kg |
| | cyclosporine | day 42-126: oral 2× weekly, IM 5× weekly |
| | | target blood levels: 4 weeks 300 ng/ml; 4 weeks 200 ng/ml; |
| | | 4 weeks 100 ng/ml |

Rhesus Anti-Chimeric Antibody (RACA) Titers

Blood samples (clotted blood) were collected at regular time points, pre- and post-transplantation from the femoral vein in the groin using aseptic techniques: Vacutainer blood collection systems (Becton Dickinson, Vacutainer systems, France) were used. Serum was collected by centrifugation and stored at −80° C. until further use.

For the determination of rhesus-anti-chimeric antibody (RACA) IgG responses against ch5D12 and chFun1, 96-well flat-bottom ELISA plates were coated with 1 μg/ml murine 5D12 or murine Fun1. Plates were incubated overnight at 4° C. or 1 hr at 37° C. with 100 ng/well and 500 ng/well to determine the IgG RACA and the IgM RACA response, respectively. The plates were washed on an automated washer and blocked with 200 μl 1% BSA (RIA grade) in PBS for 1 hr at 37° C. The plates then were emptied and incubated for 2 hrs at 37° C. with 100 μl/well of serial dilutions of the serum samples. After washing, plates were incubated with alkaline phosphatase-labeled rabbit-anti-monkey-IgG (Sigma, The Netherlands). The plates were then washed again, followed by addition of 100 μl/well substrate (p-Nitrophenyl Phosphate (pNPP)). Absorbance was measured at 405 nm. The first dilution of IgG RACA to be higher than three times the pre-transplantation value was taken as the absolute titer. IgM antibodies were expressed as index of post-transplantation values divided by pre-transplantation values, because pre-transplantation values had considerable background and inter-animal variation. The IgM RACA was considered to be positive when the index was 1.5 or higher on two or more consecutive time points.

Donor-Specific Antibodies

Blood samples (clotted blood) were collected at regular time points, pre- and post-transplantation from the femoral vein in the groin using aseptic techniques: Vacutainer blood collection systems (Becton Dickinson, Vacutainer systems, France) were used. Serum was collected by centrifugation and stored at −80° C. until further use.

Anti-donor antibodies were assessed by incubating donor spleen cells with recipient serum. Since circulating chimeric Mabs in the recipient serum bound to donor spleen cells, and this was detected by the rabbit anti-human IgG and IgM antibodies, donor spleen cells were pre-incubated for 30 min. at 4° C., with mouse anti-human 5D12 (CD40) and Fun-1 (CD86) Mabs provided by PanGenetics BV. Donor spleen cells were also pre-incubated with 50 μl 1/20 diluted rabbit anti-human Ig (DAKO, Derunark) to block aspecific antibody binding. Cells were washed with FACS buffer (0.5% BSA, 0.05% NaN₃ in PBS). Cells were then incubated with 25 μl recipient serum, at 4° C. for 30 min. Cells were washed again and incubated with rabbit anti-human IgG- or IgM-FITC F(ab′)₂ (DAKO, Denmark, dilution 1/20). Cells were washed and fixated with formaldehyde. Before analysis cells were washed to remove the formaldehyde and resuspended in PBS. Cells were analyzed on a FACScan (BD, Mountain View, Calif., USA) using standard settings for lymphocyte analysis.

Immunophenotyping—FACS Analysis

Subset analyses were performed at regular time points using whole blood in EDTA. The blood was washed with FACS buffer (PBS/BSA/NaN₃) to remove circulating free Mab present in the serum. The samples were incubated with either fluorescein isothiocyanate (FITC)-labeled ch5D12 or chFun-1 to detect in vivo coating of the cells, or with either a non-crossblocking FITC-labeled anti-CD40 Mab (clone 26, PanGenetics, BV) or phycoerthrin-labeled anti-CD86 Mab (IT2.2, Becton Dickinson PharMingen, San Diego, Calif.) to detect the percentage of positive cells for CD40 and CDD86. CD3, CD4, CD8 and CD20 positive populations were also monitored, by using clones SP34 for CD3 (BD PharMingen, CA, USA) and clones SK3, SK1, and L27 for CD4, CD8 and CD20 respectively (BD, PharMingen). A negative control was also included. The cells were incubated for 30 min. at 4° C. The red blood cells were lysed using FACS Lysing Solution (BD, CA, USA), for 10 min. at room temperature. Cells were washed 2 times and fixated using formaldehyde. Fluorescence was measured within 48 hrs. Analysis was performed using CellQuest software (BD, CA, USA). Lymphocytes were analyzed for CD40 and CD86 coating in vivo, and for CD40, CD80 and CD86 expression using CellQuest software (Becton Dickinson).

### Example 1

### Onset of Graft Rejection

The serum creatinine and urea levels of each animal were monitored because they are the first parameters to rise when kidney function is impaired, thus serving as an early indicator of graft rejection (e.g., acute rejection). However, in the week immediately post transplantation serum creatinine and urea may also be elevated due to the transplantation procedure. When the rise in serum creatinine and urea is due to the transplant rejection, electrolytes also show abnormal values.

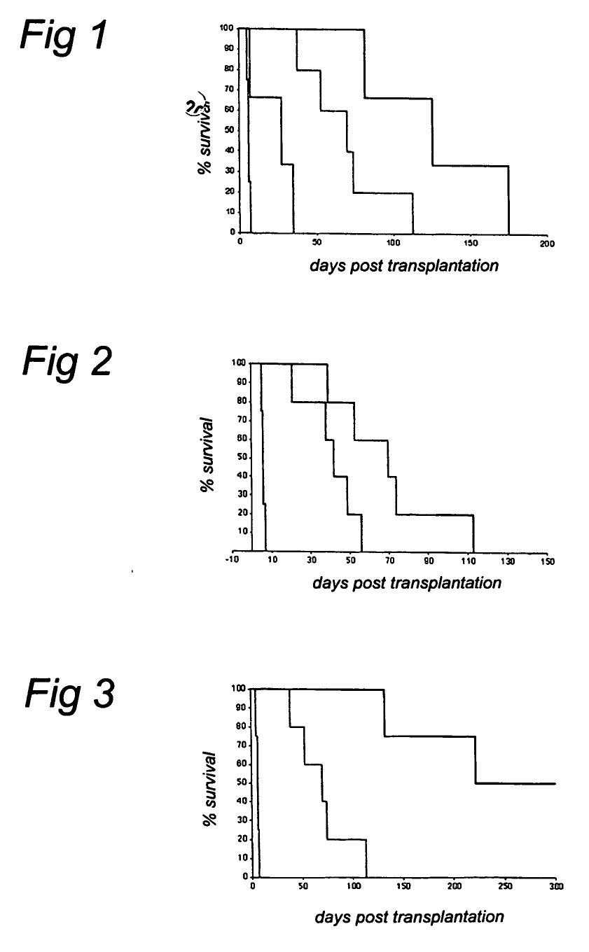

The results of this study are summarized in Table 2. The day at which the rejection process started was no different between groups 1a+b and groups 2a+b. However, it should be noted that in group 1a+b, which received anti-CD40 alone, some animals showed a short graft survival and others did not reject until several months after Mab treatment was stopped. Animals with a short graft survival which received a low dose of ch5D12 (BJG and 96087) did not show graft rejection in the kidney (C008, circulatory problem) or had a low level of circulating ch5D12 (RI075). Thus, group 1 could be subdivided into short surviving animals treated with a low level of ch5D12, and long surviving animals treated with a high level of ch5D12 (see FIG. 1). The median time to rejection was 28 days for group 1a, 126 days for group 1b and 70 days for group 2. This represents a statistically significant difference among these groups (Cox's proportional hazard analysis).

TABLE 2

| | | Day 4 creatinine | Day first significant | Day |
|---|---|---|---|---|
| Group | Animal | (μmol/liter) | rise serum creatinine | euthanized |
| 1a | BJG | 279 | 4 | 8 |
| 1b | C008 | 106 | 11 | 12 |
| 1a | 96087 | 108 | 28 | 30 |
| 1b* | RI075 | 874 | 35 | 42 |
| 1b | RI208 | 137 | 82 | 91 |
| 1b | RI149 | 125 | 126 | 134 |
| 1b | DXW | 114 | 175 | 217 |
| 2b | RI140 | 564 | 53 | 61 |
| 2a | EBP | 103 | 70 | 71 |
| 2b | RI055 | 318 | 39 | 75 |
| 2b | C118 | 129 | 74 | 78 |
| 2b | C128 | 179 | 113 | 119 |
| 3 | RI251 | 114 | 38 | 38 |
| 3 | RI286 | 410 | 21 | 56 |
| 3 | RI203 | 87 | 49 | 56 |
| 3 | RI139 | 345 | 56 | 97 |
| | RI204 | 98 | 42 | 109 |
| 4 | DKW | 97 | 133 | 141 |
| 4 | RI279 | 81 | 222 | 231 |
| 4 | RI301 | 85 | >700 | >700 |
| 4 | 97064 | 88 | >700 | >700 |

Untreated control animals rejected their grafts within one week (n = 4)

A comparison of the time to rejection of the animals in groups 2 and 3, and groups 3 and 4 are shown in FIGS. 2 and 3, respectively. The median time to rejection was 42 days for group 3, a statistically significant difference when compared to 70 days for group 2, indicating that the addition of ATG could be detrimental to graft survival when administered with the combination of Mabs.

In contrast, as shown in FIG. 3, none of the animals in group 4, which received anti-CD40+anti-CD86 followed by CsA showed a significant rise in serum creatine levels during the treatment period indicating that the median time to the onset of rejection was significantly longer in group 4 than the time to rejection in all other groups. Moreover, in at least 2 animals of group 4, serum levels remained within a normal range once treatment was terminated.

### Example 2

### Pathology of Chronic Graft Rejection

As discussed herein, chronic rejection due to continuous immune activation and subsequent tissue damage is the major problem in current transplant medicine. For this reason, kidney biopsy specimens were also taken at several time points (e.g., days 21, 42, 70) for the animals in groups 1 and 2, and compared to control animals that were treated with CsA alone (10 mg/kg i.m. daily for 35 days).

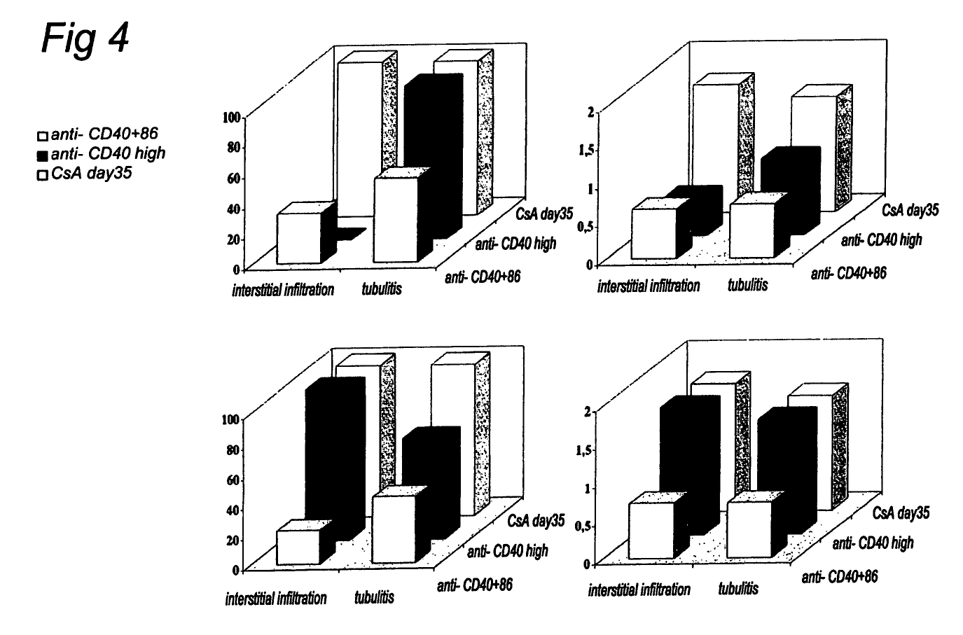

As shown in Table 3 and FIG. 4, both infiltrate scores and tubulitis scores were reduced in animals treated with anti-CD40 or anti-CD40+anti-CD86 when compared to the CsA treated controls. Moreover, on biopsies from days 21 and 42, less interstitial infiltration or tubulitis was present in animals treated with the combination of Mabs than in animals treated with ch5D12 alone. Thus, it seems that the combination of Mabs prevented graft infiltration. However, it is also possible that the infiltrating cells seen in the animals treated with ch5D12 alone were not necessarily pathogenic and may even have contained regulatory or suppressor T cells

### Example 3

### Graft Pathology after Euthanasia

Animals were euthanized before they became clinically ill due to the rejection process, and pathology was performed to determine the extent of tissue rejection. A comparison of the Banff scores for each animal is summarized in Table 3.

Of the seven animals treated with anti-CD40 alone (group 1), three rejected the transplant while still on treatment. Two of these animals received the lower dose of anti-CD40 (group 1a). One animal died after 12 days due to a blocked ureter and had only borderline signs of rejection, and the remaining three animals did not reject their graft during treatment, but at variable times after cessation of treatment.

None of the animals treated with the combination of anti-CD40 and anti-CD86 showed signs of graft rejection during treatment (group 2). However all animals rejected the kidney transplant around the end of the treatment period. Only one animal had only borderline signs of kidney rejection, but had a blocked ureter (EBP), which could also have been caused by a rejection process. The two animals that were euthanized on day 3 and 7, respectively, did not show signs of rejection. These animals were excluded from the statistical analysis. Thus, while treatment with anti-CD40 appears to result in a variable delay of graft rejection, the combination of anti-CD40 and anti-CD86 appears to completely prevent graft rejection during treatment, providing a more consistent delay in graft rejection than anti-CD40 alone.

TABLE 3

| | | | Day start | Day of | Graft pathology at necropsy: |
|---|---|---|---|---|---|
| GROUP | TREATMENT | ANIMAL | rejection | euthanasia | Acute rejection/CAN |
| 1a | anti CD40 Ld | BJG | 4 | 8 | acute rejection I-II |
| | | 96087 | 28 | 30 | acute rejection III |
| 1b | anti CD40 Hd | C008 | 11 | 12 | borderline rejection |
| | | Ri075 | 35 | 42 | acute rejection II/CAN I |
| | | Ri208 | 82 | 91 | acute rejection II/CAN III |
| | | Ri149 | 126 | 134 | acute rejection II/CAN III |
| | | DXW | 175 | 217 | acute borderline/CAN (I or) III |
| 2a | anti CD40 + | 96079 | | 7 | no rejection, CMV |
| | 86 Ld | EBP | 70 | 71 | acute rejection borderline to I |
| 2b | anti CD40 + | C146 | | 3 | PNF, no rejection |
| | 86 Hd | Ri140 | 53 | 61 | acute rejection II-III/CAN 0-I |
| | | Ri055 | 39 | 75 | acute rejection I |
| | | C118 | 74 | 78 | acute rejection I-II/CAN |
| | | C128 | 113 | 119 | acute rejection 0-I |
| 3 | anti CD40 + | Ri0271 | | 8 | PNF, no rejection |
| | 86 Hd | Ri0251 | 38 | 38 | acute rejection III |
| | ATG, steroids | Ri286 | 21 | 56 | acute rejection I |
| | CsA | Ri203 | 49 | 56 | no rejection |
| | | Ri139 | 56 | 97 | acute rejection I |
| | | Ri204 | 42 | 109 | acute rejection I/CAN II-III |
| 4 | anti CD40 + | DKW | 133 | 141 | acute rejection I/CAN I |
| | 86 Hd | Ri279 | 222 | 231 | acute rejection I/CAN I-II |
| | CsA | 97064 | | >307 | alive and well; no acute rejection/CAN I |
| | | Ri301 | | >307 | alive and well; no acute rejection/CAN II-III |

Ld = low dose;
Hd = high dose;
PNF: primary non-function of graft;
CAN: chronic allograft ephropathy;
CMV: cytomegalovirus virus infection

In group 3, in which the animals received ATG in addition to anti-CD40+86, rejection started before the end of the Mab treatment in spite of the fact that from day 42 onwards steroids and CsA were given. The median time to rejection was 42 days and this was, again, significantly different from the time to rejection in group 2 (Cox's proportional hazard analysis) (See FIG. 2.)

In contrast, as shown in FIG. 3, none of the animals in group 4, which received anti-CD40+anti-CD86 followed by CsA showed signs of transplant rejection during the treatment period. Two animals rejected after CsA treatment was stopped (one on day 141 and one on day 231), but two animals were still alive at the end of the observation period (>700 days post transplantation). Thus, the biopsies confirmed that the time to rejection was significantly longer in all animals in group 4 than the time to rejection in all other groups. Moreover, these results confirmed that long-term survival and graft-tolerance has been achieved in some animals even in the absence of continuous immunosuppressive drug treatment.

### Example 4

### Host Immune Response to the Therapeutic Mabs

The production of host antibodies against ch5D12 and chFun-1 were determined in order to evaluate the host immune response to these therapeutic Mabs.

In animals treated with anti-CD40 alone (group 1) and with anti-CD40+antiCD-86 (group 2), three animals were killed before any RACA response could be detected. Two animals from group 1, with graft survival times of 42 and 217 days, had positive anti-ch5D12 IgM RACA starting on days 13 and 11, respectively. These reactions persisted for more than a week. None of the animals from group 2 had a positive anti-ch5D12 IgM response, but three animals had rather low, but positive, anti-Fun-1 IgM indexes, all starting on day 11.

In both groups 1 and 2, a number of animals developed anti-ch5D12 IgG responses. One animal from group 1 developed a relatively low anti-ch5D12 IgG titer, more than 10 days after the last injection on day 56, and had a graft survival of 91 days. The other two animals in group 1 (graft survivals of 135 and 217 days) did not develop RACA. Animals in group 2 that rejected during treatment had high titers within 4 weeks after the start of treatment. Because of this anti-ch5D12 RACA development, these two animals had rapidly declining levels of ch5D12 (see below) and rejected early. Another two animals from group 2 developed anti-ch5D12 IgG antibodies immediately after treatment was stopped, and these animals rejected on days 71 (Grade I) and 78 (grade I-II). One animal in this group developed a lower anti-ch5D12 IgG titer and rejected on day 116. Two animals did not develop a RACA response against ch5D12, and these animals rejected on days 61 (fade II-III acute+0-−I chronic) and 75 (grade I). In general, it appears that for groups 1 and 2, animals that did not develop any anti-ch5D12 IgG titer generally survived longer than animals that did develop an anti-ch5D12 IgG RACA response.

chFun-1 levels in animals of group 3 all showed a significant drop by day 20 to 30, which could be explained by the anti-chFun-1 response present in all animals. ch5D12 levels in animals of group 3 also showed a significant drop. With the exception of Ri251, all animals were negative for an anti-ch5D12 response. Although the absorbance was increased in some post-transplantation samples, this never rose above pre-value+3×SD, with Ri251 as an exception.

Considerable anti-chFun-1 antibodies were also found in the animals of group 4. The two animals with the highest titers of this Mab in this group (97064 and Ri279) also had the lowest ch5D12 levels. This results show that chFun-1 is more immunogenic than ch5D12 in rhesus monkeys and furthermore results for group 4 show that in this group anti-chFun-1 antibody response does not induce rejection as shown by the long survival time of all animals in this group.

### Example 5

### Correlation of Therapeutic Mab Levels and Graft Survival

The serum Mab levels of the therapeutic Mabs in each animal were determined to examine the correlation of Mab concentration and graft survival.

Circulating Mab levels in these the low dose animals for groups 1a and 2a were found to be lower than 100 μg/ml serum after day 14. Therefore, the remaining animals in these groups were treated with a doubling of the dosing schedule to try and maintain circulating Mab levels above 100 μg/ml throughout the treatment period. Animals in group 1b, and one animal in group 1b that developed a RACA response against ch5D12 demonstrated ch5D12 levels of less than 100 μg/ml with rapidly declining levels thereafter. These animals rejected early (days 8, 30, 42). The rest of the animals in group 1b and in group 2 that maintained higher circulating levels of ch5D12 had longer survival rates.

Some of the animals in group 3 were found to have low Mab levels at the day 0, and in all animals, levels were already below 100 μg/ml serum by day 40. This was the case for both ch5D12 and chFun-1Mabs. The two animals that lived the longest (Ri139 and Ri204), had at least a low level of both Mabs in their blood around day 40, while the animals that lived a shorter time had almost no Mabs in their blood by day 40.

In striking contrast, the Mab levels in animals in group 4 stayed high until the end of the treatment period. However, in this group, no correlation could be found between the height of the Mab levels and the survival time. Specifically, animal 97064 had the lowest levels of both Mabs, and this animal lived longer than at least two other monkeys (DKW and Ri279).

### Example 6

### Correlation of Donor Specific Antibody Levels and Graft Rejection

Donor-specific antibodies were also measured as an indicator of graft rejection in all animals, except animal C146.

Donor-specific IgG antibodies developed in three animals of group 1 (96087, Ri149 and DXW). In all these cases the antibodies only reached significant levels at the day of rejection. Even then, the percentage of donor cells stained, was lower than 20%. Three animals of group 3 (Ri251, Ri139 and Ri203) developed donor-specific IgG antibodies. In Ri251 and Ri203 this was correlated with a short survival time. Generally, donor-specific antibodies are not known to have a detrimental effect on survival, but can be an indicator of the poor immunosuppressive state that the animals are in.

None of the animals of groups 2 and 4 developed anti-donor IgG antibodies. The anti-donor IgM antibodies were difficult to interpret because of a high background that varied per test. However, some animals (e.g., Ri140, group 3) formed donor-specific IgM antibodies, while no IgG antibodies could be detected.

### Example 7

### Immunophenotyping of Peripheral Blood Lymphocytes

To investigate the systemic effects of the Mab treatments, lymphocyte subset FACS analyses were performed at regular time points using whole EDTA blood.

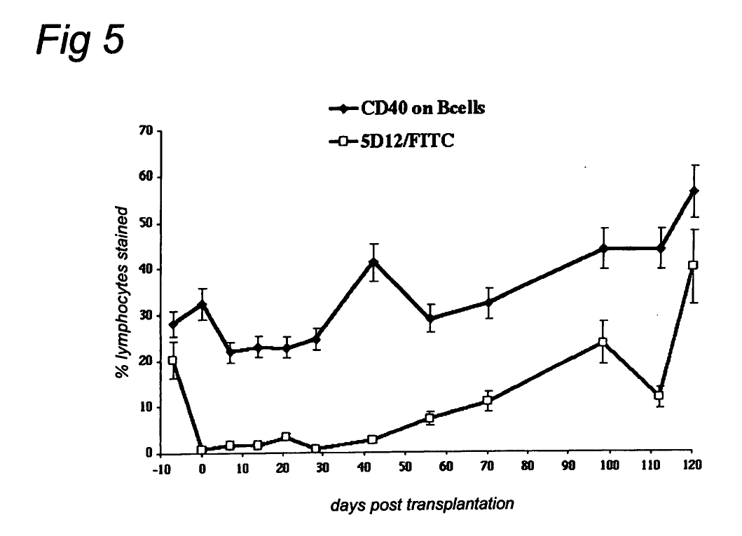

As demonstrated in FIG. 5, cells from animals of group 2 and 3 could not be stained using 5D12/FITC during treatment, but were detectable using another, non-competing anti-CD40 Mab. This indicates that CD40 was completely coated with ch5D12 in vivo, but that CD40 positive cells were not removed from the circulation, although a small decrease in CD40 positive cells can be seen from day 7 until day 28.

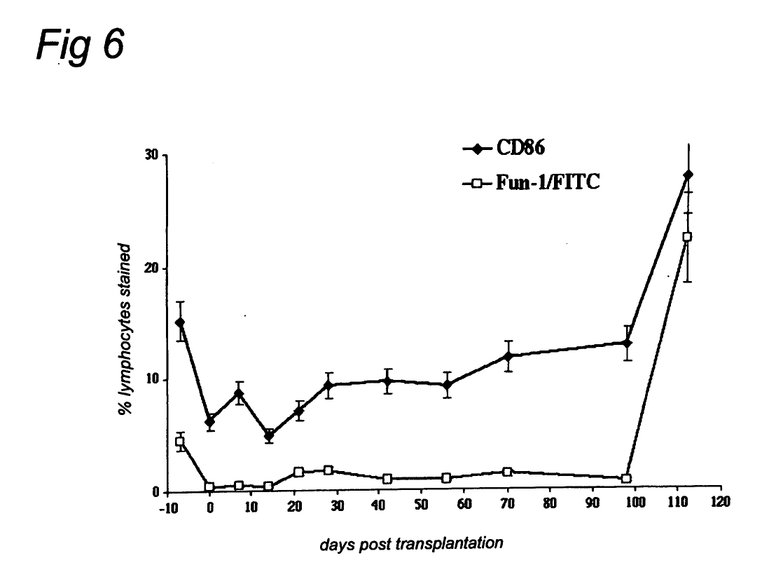

FIG. 6 shows the CD86 expression on the cells of the animals treated with the combination of ch5D12 and chFun-1. The anti-CD86 mAb stained more cells than Fun-1. As for ch5D12, no cells could be stained by Fun-1/FITC and a decrease in CD86 positive cells was observed, indicating both a complete coating of CD86 and down-regulation of the number of CD86 positive cells. CD3⁺, CD4⁺, CD8⁺, and CD20⁺ cell populations did not change during the time of treatment.

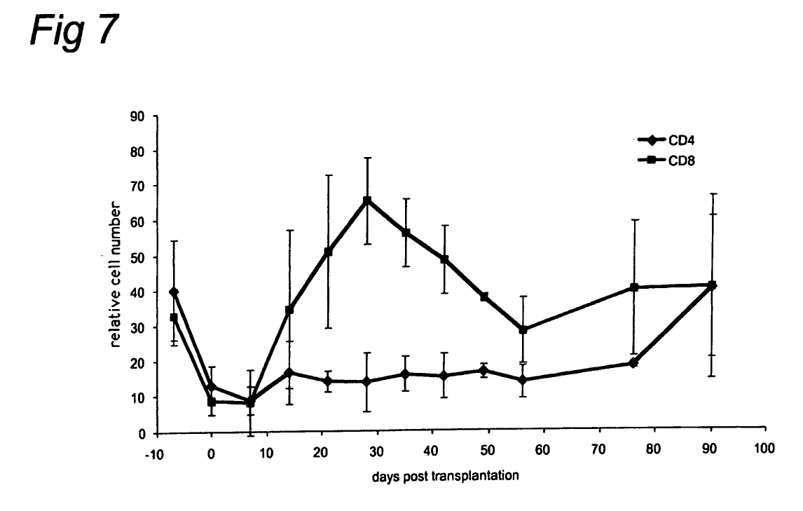

The animals in group 3, treated with ATG, showed upon the return of the lymphocytes a preferential return of CD8+ cells. This could be an explanation of the early rejection as CD8+ T cells are thought to be responsible for cytolysis while regulatory T cells are of the CD4+ phenotype (See FIG. 7).

These results clearly show full coating of both CD40- and CD86-bearing cells. Furthermore Mab treatment was without an effect on the number of various immune cells in the circulation, showing that the Mabs did not cause cell depletion. ATG pre-treatment caused depletion of T cells, showing first CD8+ re-appearance in the absence of CD4+ regulatory cells.

### Example 8

### Latent TGF-β

In this example, development of TGF-β was studied as evidence of tolerance to transplant. Torrealba et al. (2004) have found that latent TGF-β in biopsies of stable kidney graft recipients correlates with the absence of rejection and anti-donor responses in the trans-vivo DTH assay.

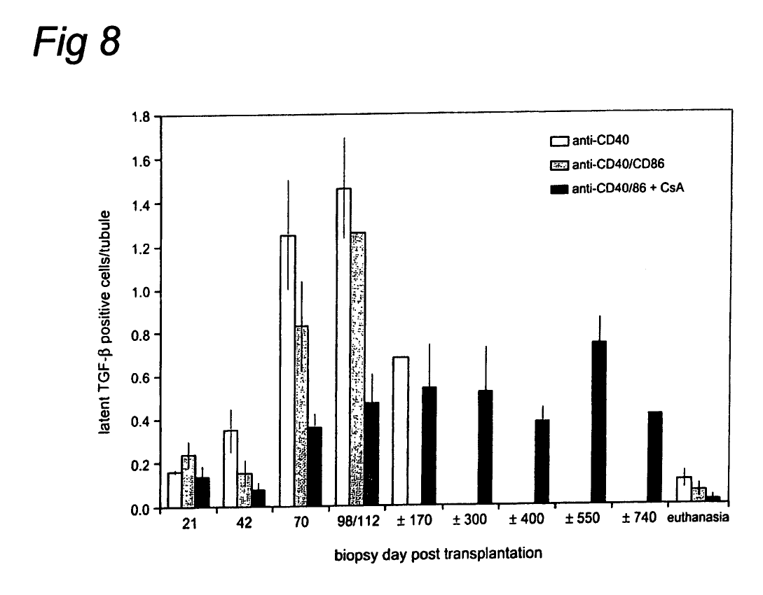

Kidney biopsies taken from monkeys in groups 1, 2, and 3 were analysed for the presence of latent TGF-β. Biopsies were taken during treatment, as well as post-treatment. Kidney biopsies were stained for latent TGF-β and scored blindly for the number of TGF-β positive areas per tubule. FIG. 8 shows mean latent TGF-β staining/tubulus per group (+/−SEM). Latent TGF-beta is absent at the time of rejection, when euthanasia is indicated. Biopsies taken during costimulation blockade also have only low amounts of latent TGF-β present in the graft. A trend can be seen that latent TGF-β expression is decreased in the group of animals that reject after cessation of treatment (group 2), as compared to group 1, while no differences in Banff rejection score could be detected between both groups. Animals of group 3 have lower amounts of latent TGF-β than animals both in groups 1 and 2 in day 70 and day 112 biopsies. The treatment with CsA seems to cause these lower levels of TGF-β, but after CsA is stopped, levels of TGF-β staining increase. The development of TGF-β staining during the post transplant period was shown by biopsies of two long-term surviving monkeys (>1130 and >1160 days). Early biopsies demonstrate a pattern of isolated areas of staining in the interstitium, while later biopsies demonstrate more widely dispersed areas of interstitial staining. The presence of TGF-β indicates that active down-regulation of immune reactivity may be one of the mechanisms by which graft rejection is prevented.

Discussion

As presented above, six different treatment regimens were tested in rhesus monkeys that underwent kidney allograft transplantation: (1) anti-CD40 low dose (Group 1a); (2) anti-CD40 high dose (Group 1b); anti-CD86 low dose (Group 2a); (4) anti-CD86 high dose (Group 2b); (5) Pre-treatment with ATG, followed by anti-CD40 and anti-CD86 high dose, and then steroids and CsA (Group 3); and (6) anti-CD40 and anti-CD86 high dose, followed by CsA (Group 4).

Treatment with anti-CD40 Mabs provided an immunosuppressive effect for kidney allografts in rhesus monkeys treated with high doses of the Mabs (group 1b). Although significant numbers of monocytes were seen in graft biopsies on days 21 and 42, no impairment of graft function was found. However, after discontinuation of Mab treatment, rejection occurred in 3 out of 4 animals (1 died of other causes), and two animals showed signs of chronic graft rejection. This is likely that indication that the blockade of CD40 has resulted in immune suppression mediated by regulatory T cells, which disappeared after discontinuation of Mab treatment.

The combination of anti-CD40 plus anti-CD86 also prevented transplant rejection during Mab treatment, although in one case the rejection process was already ongoing during the last weeks of Mab treatment. However, as with the anti-CD40 treatment, all animals rejected the kidney allografts in an acute fashion shortly after discontinuation of the treatment. Therefore, it seems that if anti-CD40 promoted the appearance of regulatory T cells, as evidenced by graft infiltrating cells, the anti-CD86 treatment may have counteracted this. This treatment is likely a more a general suppression of T-cell activation and once it is was stopped, the grafts are rejected.

The addition of ATG to the combination of anti-CD40 plus anti-CD86 treatment resulted in an even earlier rejection than when anti-CD40 plus anti-CD86 was given alone. ATG results in a rigorous depletion of T cells, both from the periphery as well as from central lymphoid tissue. This results in immunosuppression. T cells start to reappear 2 to 3 weeks after the treatment, with CD8⁺ T cells reappearing earlier that CD4⁺ T cells. This imbalance may be the cause of the earlier rejection in this group. Rather than synergizing, ATG and the blockade of co-stimulation thus appear to counteract one another, and should not be combined in protocols aiming at induction of graft prolongation where the formation of regulatory T cells is involved.

In contrast to the counteractive effects of calcineurin inhibitors on the tolerizing potential of costimulation blockade described by others (see, e.g., Kirk et al., 1999), the data presented herein demonstrate that treatment with an anti-CD40 antagonist alone or in combination with a anti-CD86 antagonist, followed by CsA treatment, not only prevented graft rejection during treatment, but achieved long-term survival and transplant tolerance in some of the subjects. In addition, the data presented herein demonstrate that by co-administering an anti-CD40 antagonist alone or in combination with an anti-CD86 antagonist, the level of immunosuppressive drugs required for maintenance therapy was lower than that used in conventional immunosuppressant therapies. For example, while two of four animals subjected to the combined antagonist and immunosuppressive drug regiment rejected their transplant after CsA was stopped, two animals have survived without a rise of serum creatinine more than 100 weeks in the complete absence of any immune suppressive maintenance therapy. Therefore, costimulation blockade followed by conventional immunosuppression significantly reduces the amount of immunosuppression needed to maintain graft survival.

### REFERENCES

1. Bontrop R E, Otting N, Slierendregt B L, Lanchbury J S. Evolution of major histocompatibility complex polymorphisms and T-cell receptor diversity in primates. Immunol Rev 1995; 143: 33-62.
2. Doxiadis G G, Otting N, de Groot N G, Noort R, Bontrop R E. Unprecedented polymorphism of Mhc-DRB region configurations in rhesus macaques. J Immunol 2000; 164 (6): 3193-9.
3. Doxiadis G G, Otting N, Antunes S G, et al. Characterization of the ABO blood group genes in macaques: evidence for convergent evolution. Tissue Antigens 1998; 51 (4 Pt 1): 321-6.
4. Fishman J A, Rubin R H. Infection in organ-transplant recipients. N Engl J Med 1998; 338 (24): 1741-51.
5. Haanstra K G, Ringers, J, Sick E A et al., Transplantation, 2003, 75 (5): 637-643.
6. Hausen B, Klupp J, Christians U, et al. Coadministration of either cyclosporine or steroids with humanized monoclonal antibodies against CD80 and CD86 successfully prolong allograft survival after life supporting renal transplantation in cynomolgus monkeys. Transplantation 2001; 72 (6): 1128-37.
7. Kenyon N S, Chatzipetrou M, Masetti M, et al. Long-term survival and function of intrahepatic islet allografts in rhesus monkeys treated with humanized anti-CD154. Proc Natl Acad Sci USA 1999; 96 (14): 8132-7.
8. Kenyon N S, Fernandez L A, Lehmann R, et al. Long-term survival and function of intrahepatic islet allografts in baboons treated with humanized anti-CD154. Diabetes 1999; 48 (7): 1473-81.
9. Kirk A D, Harlan D M, Armstrong N N, et al. CTLA4-Ig and anti-CD40 ligand prevent renal allograft rejection in primates. Proc Natl Acad Sci USA 1997; 94 (16): 8789-94.
10. Kirk A D, Burkly L C, Batty D S, et al. Treatment with humanized monoclonal antibody against CD154 prevents acute renal allograft rejection in nonhuman primates. Nat Med 1999; 5 (6): 686-93.
11. Kirk A D, Tadaki D K, Celniker A, et al. Induction therapy with monoclonal antibodies specific for CD80 and CD86 delays the onset of acute renal allograft rejection in non-human primates. Transplantation 2001; 72 (3): 377-84.
12. Knechtle S J, Harnawy M M, Hu H, Fechner Jr J H, Cho C S. Tolerance and near-tolerance strategies in monkeys and their application to human renal transplantation. Immunol Rev 2001; 183: 205-13.
13. Montgomery S P, Hale D A, Hirshberg B, Harlan D M, Kirk A D. Preclinical evaluation of tolerance induction protocols and islet transplantation in non-human primates. Immunol Rev 2001; 183: 214-22.
14. Neuhaus P, Neuhaus R, Wiersema H D, Borleffs J C, Balner H. The technique of kidney transplantation in rhesus monkeys. J Med Primatol 1982; 11 (3): 155-62.
15. Ossevoort M A, Ringers J, Kuhn E M, et al. Prevention of renal allograft rejection in primates by blocking the B7/CD28 pathway. Transplantation 1999; 68 (7): 1010-8.
16. Penn I. Post-transplant malignancy: the role of immunosuppression. Drug Saf 2000; 23 (2): 101-13.
17. Pierson R N, 3rd, Chang A C, Blum M G, et al. Prolongation of primate cardiac allograft survival by treatment with ANTI-CD40 ligand (CD154) antibody. Transplantation 1999; 68 (11): 1800-5.
18. Racusen L C, Solez K, Colvin R B, et al. The Banff 97 working classification of renal allograft pathology. Kidney Int 1999; 55 (2): 713-23.
19. Torrealba J R, Katayama M, Fechner J H, Jr., et al. Metastable tolerance to rhesus monkey renal transplants is correlated with allograft TGF-beta 1+CD4+T regulatory cell infiltrates. J Immunol 2004; 172 (9): 5753.

1-25. (canceled)

26. A method of inducing tolerance to a transplant in a subject comprising:
(a) administering multiple doses of a therapeutically effective amount of a CD40 antagonist, alone or in combination with a CD86 antagonist, wherein the first dose of antagonist is given before or at the time of transplantation; and
(b) administering multiple doses of a therapeutically effective amount of an immunosuppressive drug, wherein the first dose of the immunosuppressive drug is given at least about 2 days after transplantation.

27. The method of claim 26, wherein the CD40 antagonist is administered for a period sufficient to tolerize T cells to the transplant.

28. The method of claim 26, wherein the CD40 antagonist is administered for a period of at least about 6-12 weeks.

29. The method of claim 26, wherein the CD40 antagonist is administered in an amount sufficient to achieve a serum level of at least about 10-300 μg/ml.

30. The method of claim 26, further comprising administering an antagonist ex vivo to the transplant prior to transplantation.

31. The method of claim 26, wherein the first dose of the immunosuppressive drug is administered at least about 5 days after transplantation.

32. The method of claim 26, wherein the first dose of the immunosuppressive drug is administered at least about 2 weeks after transplantation.

33. The method of claim 26, wherein the first dose of the immunosuppressive drug is administered following completion of administration of the antagonist.

34. The method of claim 26, wherein the first dose of the immunosuppressive drug is administered upon the onset of transplant rejection.

35. The method of claim 26, wherein the immunosuppressive drug is administered for a period until tolerance to the transplant is achieved in the absence of the antagonist or the immunosuppressive drug.

36. The method of claim 26, wherein the immunosuppressive drug is administered for a period of at least about 4-12 weeks.

37. The method of claim 26, wherein the antagonist and the immunosuppressive drug are administered in tapering dosages.

38. The method of claim 26, wherein administering antagonist in step (a) comprises administering a combination of the CD40 antagonist and the CD86 antagonist.

39. The method of claim 26, wherein the antagonist is selected from the group consisting of an antibody, a bispecific antibody, a soluble receptor, a peptide and a small molecule.

40. The method of claim 39, wherein the antibody is selected from the group consisting of a chimeric antibody, a humanized antibody and a human antibody.

41. The method of claim 26, wherein the CD40 antagonist is ch5D12.

42. The method of claim 38, wherein the CD40 antagonist is ch5D12 and the CD86 antagonist is chFun-1.

43. The method of claim 26, wherein the immunosuppressive drug is a signal 1 blocker.

44. The method of claim 26, wherein the immunosuppressive drug is selected from the group consisting of cyclosporine, tacrolimus, azathioprine, a corticosteroid, mycophenolate mofetil, rapamycin, OKT3 and an anti-CD25 antibody.

45. The method of claim 26, wherein the immunosuppressive drug is cyclosporine A.

46. The method of claim 26, wherein the transplant is an allograft.

47. The method of claim 26, wherein the transplant is an organ.

48. A method of inducing tolerance to a transplant in a subject comprising:
(a) administering a therapeutically effective amount of a CD40 antagonist, alone or in combination with a CD86 antagonist, over a period of at least about 4-10 weeks, wherein the first dose of antagonist occurs before or at the time of transplantation; and
(b) administering a therapeutically effective amount of an immunosuppressive drug over a period sufficient to achieve tolerance to the transplant in the absence of antagonist or the immunosuppressive drug, wherein the first dose of the immunosuppressive drug occurs no sooner than about 4 weeks after transplantation.

49. A method of inducing tolerance to a transplant in a subject comprising:
(a) administering multiple doses of a therapeutically effective amount of a CD40 antagonist, alone or in combination with an antagonist of a second costimulatory molecule, wherein the first dose of antagonist is given before or at the time of transplantation; and
(b) administering multiple doses of a therapeutically effective amount of an immunosuppressive drug, wherein the first dose of the immunosuppressive drug is given at least about 2 days after transplantation.

50. The method of claim 49, wherein the antagonist of the second costimulatory molecule is a CD86 antagonist.
